(19) United States
(12) Patent Application Publication
(10) Pub. No.: US 2011/0226620 A1
(43) Pub. Date: Sep. 22, 2011

(54) Highly Homogeneous Molecular Markers for Electrophoresis

(75) Inventors: Mitra Tadayoni-Rebek, La Jolla, CA (US); Joseph W. Amshey, Encinitas, CA (US); Regina Rooney, La Jolla, CA (US)

(73) Assignee: LIFE TECHNOLOGIES CORPORATION, Carlsbad, CA (US)

(21) Appl. No.: 12/786,406

(22) Filed: May 24, 2010

Related U.S. Application Data
(63) Continuation of application No. 11/840,164, filed on Aug. 16, 2007, now abandoned.
Application No. 11/840,164 is a continuation of application No. 10/369,117, filed on Feb. 20, 2003, now abandoned.
Application No. 10/369,117 is a continuation-in-part of application No. 09/927,436, filed on Aug. 13, 2001, now abandoned.
(60) Provisional application No. 60/357,634, filed on Feb. 20, 2002.
(60) Provisional application No. 60/224,345, filed on Aug. 11, 2000.

(51) Int. Cl. G01N 27/447; C07K 7/08; C07K 14/245; C07K 1/13; C25B 7/00

(52) U.S. Cl. 204/450; 530/324; 530/327; 530/350; 530/326; 530/402; 530/345

(57) ABSTRACT

The invention relates to marker molecules for identifying physical properties of molecular species separated by the use of electrophoretic systems. The invention further relates to methods for preparing and using marker molecules.

### CROSS-REFERENCE TO RELATED APPLICATIONS

This application is a continuation and claims the right of priority under 35 U.S.C. §120 to U.S. Ser. No. 11/840,164, filed Aug. 16, 2008, now abandoned, which is a continuation application of U.S. Ser. No. 10/369,117, abandoned, filed Feb. 20, 2003, which claims the benefit of priority under 35 U.S.C. §119 of U.S. Ser. No. 60/357,634, filed Feb. 20, 2002; U.S. Ser. No. 09/927,436, filed Aug. 13, 2001 and U.S. Ser. No. 60/224,345, filed Aug. 11, 2000, the entire contents of each of which are incorporated herein by reference.

### BACKGROUND OF THE INVENTION

1. Field of the Invention

The present invention is in the fields of molecular biology and protein biochemistry. The invention relates to marker molecules for identifying physical properties of molecular species separated by the use of electrophoretic systems. The invention further relates to methods for preparing and using marker molecules.

2. Background Art

Gel electrophoresis is a common procedure for the separation of biological molecules, such as deoxyribonucleic acid (DNA), ribonucleic acid (RNA), polypeptides and proteins. A common method of electrophoresis of proteins involves equilibrating the sample with a negatively-charged surfactant such as sodium dodecylsulfate (SDS) before electrophoresis. This causes all the proteins to have a net negative charge and thus migrate toward the anode. Nucleic acids are charged without further change. In gel electrophoresis, the molecules are separated into bands according to the rates at which an imposed electric field causes them to migrate through a medium.

A commonly used variant of this technique consists of an aqueous gel enclosed in a glass tube or sandwiched as a slab between glass or plastic plates. The gel has an open molecular network structure, defining pores that are saturated with an electrically conductive buffered solution of a salt. These pores through the gel are large enough to admit passage of the migrating macromolecules.

The gel is placed in a chamber in contact with buffer solutions which make electrical contact between the gel and the cathode or anode of an electrical power supply. A sample containing the macromolecules and a tracking dye is placed on top of the gel. An electric potential is applied to the gel causing the sample macromolecules and tracking dye to migrate toward one of the electrodes depending on the charge on the macromolecule. The electrophoresis is halted just before the tracking dye reaches the end of the gel. The locations of the bands of separated macromolecules are then determined. By comparing the distance moved by particular bands in comparison to the tracking dye and macromolecules of known mobility, the mobility of other macromolecules can be determined. The size of the macromolecule can then be calculated or macromolecules of different sizes can be separated in the gel.

Isoelectric focusing (IEF) is an electrophoresis method based on the migration of a molecular species in a pH gradient to its isoelectric point (pI). The pH gradient is established by subjecting an ampholyte solution containing a large number of different-pI species to an electric field, usually in a cross-linked matrix such as a gel. Analytes added to the ampholyte-containing medium will migrate to their isoelectric points along the pH gradient when an electrical potential difference is applied across the gel.

For complex samples, multidimensional electrophoresis methods have been employed to better separate species that co-migrate when only a single electrophoresis dimension is used. Common among these is two dimensional electrophoresis or 2D-E. For 2D-E analysis of proteins, for example, the sample is usually fractionated first by IEF in a tube or strip gel to exploit the unique dependence of each protein's net charge on pH. Next, the gel containing the proteins separated by pI is extruded from the tube in the case of a tube gel, equilibrated with SDS and laid horizontally along one edge of a slab gel, typically a cross-linked polyacrylamide gel containing SDS. Other methods for IEF fractionation allow pieces or strips of gel supported on non-conductive backing to be laid directly onto the slab of gel. Electrophoresis is then performed in the second dimension, perpendicular to the first, and the proteins separate on the basis of molecular weight. This process is referred to as SDS polyacrylamide gel electrophoresis or SDS-PAGE. The rate of migration of macromolecules through the SDS-PAGE gel depends upon four principle factors: the porosity of the gel; the size and shape of the macromolecule; the field strength; and the charge density of the macromolecule. It is critical to an effective electrophoresis system that these four factors be precisely controlled and reproducible from gel to gel and from sample to sample. However, maintaining uniformity between gels is difficult because each of these factors is sensitive to many variables in the chemistry of the gel and the other reagents in the system as well as the characteristics of the macromolecules. Thus, proteins having similar net charges, which are not separated well in the first dimension (IEF), will separate according to variations of the other principle factors in the second dimension (SDS-PAGE). Since these two separation methods depend on independent properties, the overall resolution is approximately the product of the resolution in each dimension.

Essential to the practice of many of these electrophoretic techniques, including 2D-E and SDS-PAGE, are molecular marker standards, i.e. standard protein molecules with known molecular weights and pIs. Molecular markers are used as benchmarks in electrophoresis systems for comparison of physical properties with the unknown samples of interest. Although there are numerous applications for molecular markers, some particular examples include: conventional two-dimensional gel electrophoresis using broad pH range immobilized pH gradient (IPG) strips, overlapping two-dimensional gel electrophoresis using narrow pH range IPG strips, stand-alone SDS-PAGE, IEF gels with carrier ampholytes, capillary electrophoresis, electrokinetic chromatography. Many other forms of gel electrophoresis are well known to those of skill in the art.

Thus, it is desirable to have reliable standard markers with well-defined properties with which to compare an unknown sample. This is particularly true in high-resolution systems such as 2D-E. Unfortunately, commercially available 2D-E standards (BioRad, Hercules, Calif., Catalogue No. 161-0320; Sigma, St. Louis, Mo., Catalogue No. G0653; Pharmacia, Uppsala, Sweden, Catalogue Nos. 17-0471-01 and 17-0582-01) consist mainly of unstained natural proteins that are only available in a limited range of pIs and molecular weights. These commercial markers randomly distribute on two-dimensional gels and cannot be distinguished from the analyte. Furthermore, manipulation of pI and molecular weight of proteins using various agents generates a heterogeneous mixture of products that do not migrate in a sharp zone under electrophoretic conditions. This is particularly a problem when using conventional techniques to make proteins visibly detectable by attaching chromophoric groups. In the current state of the art, proteins are labeled by treating the protein with a reactive agent which may be a chromophoric group or other label. Since the protein has multiple potentially reactive sites such as —NH₂ or —SH groups, and since complete reaction of all sites is never achieved, the labeling reaction results in a mixture of products. A single population of markers may have varying numbers of labels depending on how many active sites are available. This heterogeneous mixture of molecules will vary in pIs and molecular weights and will produce smeared or diffused bands or spots under electrophoretic conditions. Lack of precision for molecular markers will have a negative effect on all separation techniques, especially those involving isoelectric focusing. The smearing or blurred appearance of the markers during visualization of the results will lead to ambiguous or unreliable representation of the experimental data. Consequently, there is an unmet need for highly homogeneous visible molecular markers that are compatible with commercially available separation techniques, especially techniques that separate proteins on the basis of charge and/or molecular weight.

### SUMMARY OF THE INVENTION

The present invention is directed to methods for preparing homogeneous visible, preferably colored marker molecules with known pIs and molecular weights. The invention is further directed to methods of altering the pI and molecular weight of proteins or nucleic acids in a consistent, reproducible fashion using organic molecules or peptides. Marker molecules of the present invention will generally separate to give narrow, sharp bands or spots under electrophoretic conditions. The present invention is also directed to methods of preparing marker molecules of the present invention and methods for using these molecules.

In one embodiment, the present invention relates to marker molecule compositions comprising same pI and same molecular weight marker molecules. In another embodiment, the present invention relates to marker molecule compositions comprising same pI and different molecular weight marker molecules. In yet another embodiment, the present invention relates to marker molecule compositions comprising different pI and different molecular weight marker molecules. In a further embodiment, the present invention relates to marker molecule compositions comprising different pI and same molecular weight.

In another embodiment, the present invention relates to a marker molecule comprising: a molecular weight from about 200 daltons to about 2,000 daltons, from about 300 daltons to about 2,500 daltons, from about 3,000 daltons to about 250,000 daltons, an isoelectric point (pI) from about 2 to about 12, and at least one or more labeling molecules. Such labeling molecules may include chromophores, fluorophores, or ultraviolet light (UV) absorbing groups. Labeling may also be achieved by introducing natural amino acids containing UV absorbing moieties such as the aromatic groups in tryptophan and tyrosine (Shimura, K. et al., Electrophoresis 21:603-610 (2000)). In another embodiment, the present invention relates to a marker molecule of the formula:

Segment A-L-Segment B

wherein,
Segment A is a labeled molecule (e.g., natural or synthetic, including, without limitation, organic molecules, polypeptide, polynucleotides, macromolecule such as carbohydrates, small molecules, oligopeptides, natural or non-natural amino acids), preferably labeled with one or more chromophores, fluorophores, or UV absorbing groups;
L is a linker or a bond;
Segment B is a protein (e.g., native, recombinant or synthetic protein) or nucleic acid (e.g., DNA or RNA).

In a further embodiment, the present invention relates to marker molecule compositions comprising a collection of two or more (e.g., one, two, three, four, five, six, seven, eight, nine, ten, fifteen, twenty, etc.) marker molecules of the present invention wherein the marker molecules differ in molecular weight and/or isoelectric point (pI).

In another embodiment, the present invention relates to marker molecules wherein the labeling molecules are selected from the group consisting of chromophores, fluorophores, and UV absorbing groups.

In a further embodiment, the present invention relates to the use of marker molecules of the present invention in gel electrophoresis systems (eg., two-dimensional gel electrophoresis systems).

In another embodiment, the present invention relates to methods of separating one or more proteins present in a sample by gel electrophoresis, comprising adding the marker molecule composition of the present invention to the sample containing one or more proteins, applying the sample to an electrophoresis gel, and subjecting the electrophoresis gel to an electric field.

In a further embodiment, the present invention relates to methods further comprising detecting one or more marker molecules and comparing the position of one or more marker molecules to the position of the one or more proteins after subjecting the gel to an electric field. In yet another embodiment, the present invention relates to methods of separating one or more proteins present in a sample by using two-dimensional gel electrophoresis.

In yet another embodiment, the present invention relates to methods of separating one or more molecules present in a sample, comprising adding the marker molecule composition of the present invention to the sample containing one or more molecules, applying the sample to a matrix, and separating the one or more molecules.

In another embodiment, the present invention relates to a method of preparing marker molecule comprising:
(a) labeling a molecule (e.g., a polypeptide of known molecular weight); and
(b) ligating the molecule with a protein or nucleic acid (e.g., a protein or nucleic acid of known molecular weight), wherein the molecule or protein (or nucleic acid) contains an α-thioester and the other contains a thiol-containing moiety.

In yet another embodiment, the present invention relates to a method of preparing marker molecule compositions further comprising:
(c) repeating (a)-(b) one or more times to obtain a number of labeled marker molecules of different molecular weights and pIs; and
(d) combining the labeled marker molecules having different molecular weights and pIs.

In one embodiment, the number of labels attached to the marker molecule is known. In a further embodiment, the number of labels is at least one and will generally be one or more (e.g., one, two, three, four, five, etc.). Labels such as charged chromophoric groups may alter the pI of the final marker molecule. Chromophores with a sulfonic acid group (pKa of 1.5) will shift the pI of the marker molecule to acidic pH or chromophores with amino groups will shift the pI to basic pH. Therefore, the pI may be manipulated and as a result, marker molecules of known pI may be prepared. In yet another embodiment, the collection of marker molecules is at least more than one, preferably at least two or more (e.g., two, three, four, five, etc.).

In a further embodiment, the present invention relates to a method of preparing a marker molecule comprising:
(a) labeling a molecule, preferably a molecule of known molecular weight, comprising an amino-terminal cysteine residue; and
(b) ligating the molecule with a protein or nucleic acid of known molecular weight and comprising an C_{α}-thioester.

In yet another embodiment, the present invention relates to a method of preparing a marker molecule composition further comprising:
(c) repeating (a)-(b) one or more times to obtain a number of labeled marker molecules of different weights and pIs; and
(d) combining the labeled marker molecules of different weights and pIs.

In a further embodiment, the present invention relates to a method of labeling a marker molecule comprising:
(a) attaching a first amino acid to a solid phase;
(b) coupling said first amino acid to a second amino acid protected by blocking groups resulting in a chain of amino acids, wherein said blocking groups are removed before the addition of amino acids;
(c) extending the length of the chain by solid phase synthesis with additional amino acids, wherein said chain comprises at least one labeled amino acid, resulting in a labeled oligopeptide;
(d) releasing the labeled oligopeptide from the solid phase; and
(e) ligating the labeled oligopeptide with a protein of known molecular weight.

In one embodiment, one, two or more (e.g., two, three, four, five, etc.) additional amino acids are modified with a label. Preferably, the blocking groups are selected from the group consisting tert-butyloxycarbonyl (BOC), 9-fluorenylmethoxycarbonyl (FMOC) and their derivatives thereof.

In yet another embodiment, the present invention relates to a method of characterizing one or more proteins, comprising:
(a) electrophoresing one or more proteins (e.g., one, two, three, four, five, six, eight, ten, etc.) in a gel with at least one (e.g., one, two, three, four, five, six, eight, ten, etc.) marker molecule of the present invention;
(b) comparing the migration of the one or more proteins with the migration of the at least one marker molecule of the present invention; and
(c) optionally, determining the isoelectric point (pI) and/or molecular weight of the one or more proteins.

In a further embodiment, the present invention relates to a method of characterizing one or more molecules, comprising:
(a) separating one or more molecules (e.g., one, two, three, four, five, six, eight, ten, etc.) in a matrix with at least one (e.g., one, two, three, four, five, six, eight, ten, etc.) marker molecule of the present invention;
(b) comparing the migration of the one or more molecules with the migration of the at least one marker molecule of the present invention; and
(c) optionally, determining the isoelectric point (pI) and/or molecular weight of the one or more molecules.

In yet another embodiment, the present invention relates to a method of characterizing one or more molecules, comprising:
(a) electrophoresing one or more molecules (e.g., one, two, three, four, five, six, eight, ten, etc.) in a matrix with at least one (e.g., one, two, three, four, five, six, eight, ten, etc.) marker molecule of the present invention;
(b) comparing the migration of the one or more molecules with the migration of the at least one marker molecule of the present invention; and
(c) optionally, determining the isoelectric point (pI) and/or molecular weight of the one or more molecules.

In one embodiment, two-dimensional gel electrophoresis may be used to analyze one or more proteins to determine their molecular weights and/or pIs. In another embodiment, the marker molecule may contain at least one (e.g., one, two, three, four, five, etc.) labeled protein, preferably at least two (e.g., two, three, four, five, etc.) labeled proteins of the present invention.

In another embodiment, the present invention relates to a peptide having the formula:

Cys-Yₙ—Z

where,
Y is one or more amino acid selected from the group consisting of alanine, arginine, aspartic acid, asparagine, cysteine, glutamic acid, glutamine, glycine, histidine, iso-leucine, leucine, lysine, methionine, phenylalanine, proline, serine, threonine, tryptophan, tyrosine and valine or
any non-natural amino acid with appropriate functionality, without limitation, trans-4-hydroxyproline, 3-hydroxyproline, cis-4-fluoro-L-proline, dimethylarginine, and homocysteine; wherein at least one amino acid is labeled with a chromophore, fluorophore, or UV absorbing group, in many instances at least two (e.g., two, three, four, five, etc.) amino acids are labeled;
Z is a C-terminal amino acid (the C_{α}-carboxyl group may be modified to have an amide function) or non-natural amino acid; and
n=1-100 covalently linked amino acid(s). In one embodiment, Y may be a non-natural amino acid which is not one of the twenty amino acids commonly found in proteins. Further, as one skilled in the art would recognize, Y can be composed of different amino acids (e.g., amino acids listed above). In another embodiment, Z may be any amino acid listed above including non-natural amino acids listed above.

In another embodiment, the present invention is directed to a method of ligating nucleic acids to oligopeptides. For example, incorporation of a thiol-containing group (e.g., 1-amino-2-mercaptoethyl) into one terminus of the nucleic acid (e.g., nucleic acid-CH(NH₂)—CH₂—SH) and subsequent ligation with an oligopeptide containing C_{α}-thioester forms nucleic acid-oligopeptide conjugate. This method may be used, for example, for the construction of nucleic acid markers. Ligation of nucleic acid-CH(NH₂)—CH₂—SH with a labeled macromolecule or a labeled small organic molecule containing C_{α}-thioester may be used to form a labeled nucleic acid.

Kits serve to expedite the performance of, for example, methods of the invention by providing multiple components and reagents packed together. Further, reagents of these kits can be supplied in pre-measured units so as to increase precision and reliability of the methods. Kits of the present invention will generally comprise a carton such as a box; one or more containers such as boxes, tubes, ampules, jars, or bags; one or more (e.g., one, two, three, etc.) pre-casted gels and the like; one or more (e.g., one, two, three, etc.) buffers; and instructions for use of kit components.

In another embodiment, the present invention relates to marker molecule kits comprising a carrier having in close confinement therein at least one (e.g., one, two, three, four, five, etc.) container where the first container comprises at least one (e.g., one, two, three, four, five, six, seven, eight, nine, ten, fifteen, twenty, etc.) marker molecule of the present invention. In yet another embodiment, the marker molecule kit of the present invention further comprises instructions for use of kit components. In a further embodiment, the marker molecule kit of the present invention further comprises one or more (e.g., one, two, three, etc.) pre-casted electrophoresis gels.

In another embodiment, the present invention relates to a marker molecule of the formula I:

Segment A-L-Segment B

wherein,
Segment A is a labeled molecule;
L is a linker or a bond; and
Segment B is a protein which contains no deamidation sites. In another embodiment, the present invention relates to marker molecules wherein Segment B is a protein which contains no arginine or glutamine residues. In yet another embodiment, the present invention relates to marker molecules wherein the protein comprises an amino acid sequence selected from the group consisting of:
(a) the amino acid sequences shown in SEQ ID NO:11;
(b) the amino acid sequences shown in SEQ ID NO:12;
(c) the amino acid sequences shown in SEQ ID NO:13;
(d) the amino acid sequences shown in SEQ ID NO:14;
(e) the amino acid sequences shown in SEQ ID NO:15;
(f) the amino acid sequences shown in SEQ ID NO:16;
(g) the amino acid sequences shown in SEQ ID NO:17; and
(h) the amino acid sequences shown in SEQ ID NO:18.

In another embodiment, the present invention relates to a marker molecule of formula I:

Segment A-L-Segment B

wherein,
Segment A is a labeled molecule;
L is a linker or a bond; and
Segment B is modified naturally occurring protein which contains a reduced number of post-translational modification sites. In yet another embodiment, the present invention relates to a marker molecule wherein the post-translational sites are selected from the group consisting of:
(a) deamidation sites;
(b) glycosylation sites; and
(c) phosphorylation sites.
   In yet another embodiment, the present invention relates to marker molecules wherein in which contains none of one or more amino acid selected from the group consisting of:
(a) asparagine;
(b) glutamine;
(c) proline;
(d) serine;
(e) threonine;
(f) tyrosine; and
(g) aspartic acid.

Other embodiments of the invention will be apparent to one of ordinary skill in light of what is known in the art, the following drawings and description of the invention, and the claims.

### BRIEF DESCRIPTION OF THE FIGURES

FIG. 1 depicts a scheme showing solid phase synthesis of a peptide to be used as Segment A of the marker molecules of the invention. In this example, a resin linker is present which contains a thioester-linked glycine. Further, a N^{α}-Fmoc-Nε-TMR-Lysine is used as a building block amino acid that is labeled with tetramethylrhodamine (TMR). The N-terminal amino acid is an iminobiotin labeled glycine. The labeled peptide is released from the solid phase by treatment with benzylthiol (Ph-CH₂—SH) and the product peptide is purified by reverse phase HPLC (RP-HPLC).

FIG. 2 depicts a scheme showing a ligation of Segment A (TMR- and biotin-labeled peptide) to a protein containing N-terminal cysteine (Segment B). Upon transthioesterification of the thioester with the cysteine thiol, a S→N acyl shift takes place to generate a ligated product with the two segments, now connected by an amide bond; resulting in the generation of a final product which is a labeled protein of known molecular weight and pI.

FIGS. 3A and 3B depict schemes showing preparation of a TMR-labeled protein by coupling an organic thioester labeled with a fluorescent dye such as tetramethylrhodamine (Segment A) to a protein with N-terminal cysteine (Segment B). FIG. 3A depicts a scheme for forming a labeled protein by acylating triethylenetetramine (TREN, available from Aldrich, Milwaukee, Wis., Catalogue No. 90462) with 3.5 equiv. of an activated ester of carboxytetrarhodamine (TMR), available from Molecular Probes, OR (Catalogue No. e-6123), to form (TMR)₃-TREN 5. Acylation of N^{α}-Fmoc-Lysine with 2-iminobiotin-N-hydroxysuccinimide ester (Biotin-NS ester) yields N_{ε}-Fmoc-N^{α}-biotin-Lysine 6. Deblocking of the α-amino group of 6 followed by acylation with bromoacetyl chloride forms N_{ε}-bromoacetamido-N^{α}-biotinyl-Lysine 8. The carbodiimide coupling of 8 with α-toluenethiol results in 9. The alkylation of 5 with the thioester 9 in the presence of sodium iodide generates the quaternary ammonium salt 10 (Segment A) that upon coupling with Segment B under the same conditions described above affords 11 (chromophore to protein ratio=3). FIG. 3B depicts a scheme for forming a TMR-labeled protein by first preparing a thiol benzyl ester (13). Deprotection of the amino group of 13 in the presence of trifluoroacetic acid, 14, followed by coupling to N-hydroxy succinimidyl ester of TMR generates the benzyl thioester derivative of N-TMR-8-heptanoic acid 15. The reaction of the thioester 15 (Segment A) with recombinant protein with N-terminal cysteine (Segment B) forms TMR-protein 16 (chromophore to protein ratio=1) that can be purified by dialysis.

FIG. 4 shows solid phase synthesis of a peptide labeled with TMR (Segment A). The resin linker is a thioester-linked histidine and N^{α}-Fmoc-ε-TMR-Lysine is the building block amino acid labeled with TMR. In this scheme, the N-terminal amino acid is cysteine. After treatment with trifluoroacetic acid (TFA), the resulting product is an oligopeptide labeled with the chromophore, TMR, and tagged with the metal affinity binding (histidine)₆ sequence.

FIG. 5 depicts a scheme showing the labeling of a protein via in vitro chemical ligation. In this method, a recombinant protein with C-terminal thioester (Segment B) ligates to a TMR-labeled, polyhistidine-tagged peptide (Segment A) with N-terminal cysteine in the presence of toluene thiol, benzylthiol and thiophenol. The reaction results in a product of known molecular weight and pI.

FIG. 6 depicts a scheme showing site-specific modification of a protein that contains an N-terminal threonine or cysteine. The amino and hydroxyl groups on adjacent carbons of an N-terminal amino acid can be readily oxidized to form a protein with N-terminal aldehyde (17, Segment B). Coupling of Segment B to 19 (Segment A) results in a visibly colored protein (21) with known molecular weight and pI.

FIG. 7 depicts a scheme showing solid phase synthesis of a peptide with N-terminal cysteine (Segment A) using Fmoc-PAL-PEG-PS resin or any amide resin as described by Schnolzer, M. et al., Intl. J. Peptide Protein Research 40:180 (1990).

FIG. 8 depicts a scheme illustrating labeling of a protein via in vitro chemical ligation. In this method a recombinant protein, MBP-95aa (a 95 amino acid segment of Maltose Binding Protein) with a C-terminal thioester (Segment B) ligates to a TMR-labeled peptide with N-terminal cysteine.

FIG. 9 depicts a scheme illustrating in vitro chemical ligation using a peptide without N-terminal cysteine. The N^{α}-(1-phenyl-2-mercaptoethyl) auxiliary is coupled to the oligopeptide N-terminus using solid phase peptide synthesis. Upon ligation, the auxiliary group is removed under mild conditions.

FIG. 10 is a photograph of a NU-PAGE® 4-12% Bis-Tris gel characterizing MBP-110aa-(TMR)₂. Lane 1 is the Multimark (Invitrogen Corporation, Carlsbad, Calif.) protein marker. Lane 2 is reaction mixture containing MBP-110aa-(TMR)₂ (highest molecular weight), MBP-95aa, unreacted Cys-Leu-Lys(TMR)-Asp-Ala-Leu-Asp-Ala-Leu-Asp-Ala-Leu-Lys(TMR)-Asp-Ala-amide (lowest band) (SEQ ID NO:3). Lane 3 is blank. Lane 4 is reaction mixture containing MBP-110aa-(TMR)₂ (highest molecular weight), MBP-95aa, unreacted Cys-Leu-Lys(TMR)-Asp-Ala-Leu-Asp-Ala-Leu-Asp-Ala-Leu-Lys(TMR)-Asp-Ala-amide (SEQ ID NO:3). Lane 5 is MBP-95 aa.

### DETAILED DESCRIPTION OF THE INVENTION

Generally, when proteins are modified by the addition of specific labels to produce marker molecules for gel electrophoresis systems, the proteins are typically linked to the labels in a manner which results in the production of a mixture of products. These product mixtures typically contain molecules having various pIs and molecular weights and often smear under electrophoretic conditions. Further, the molecules lack the precision or uniformity required for molecular markers especially when such markers are to be separated by their isoelectric points. Therefore, methods for preparing marker molecules should result in the incorporation of a chromophore or other detectable group (e.g., a visibly colored molecule) in the marker molecules in such a way as to direct the label onto a single site (e.g., at one amino acid) or at a small number of locations (e.g., one, two, three, four, or five locations) rather than randomly.

The present invention relates to a marker molecule comprising:

Segment A-L-Segment B

wherein,
Segment A is a labeled molecule (e.g., natural or synthetic, including, without limitation, organic molecules, polypeptide, polynucleotides, macromolecule such as carbohydrates, small molecules, oligopeptides, natural or non-natural amino acids), preferably labeled with one or more chromophores, fluorophores, or UV absorbing groups;
L is a linker or a bond;
Segment B is a protein (e.g., native, recombinant or synthetic protein) or nucleic acid (e.g., DNA or RNA, polynucleotide). For example, Segment B may be a protein of known molecular weight (e.g., a protein having a molecular weight from about 200 daltons to about 2,000 daltons, from about 300 daltons to about 2,500 daltons, from about 1,000 daltons to about 250,000 daltons, from about 2,000 daltons to about 250,000 daltons, from about 3,000 daltons to about 250,000 daltons, from 1,000 daltons to about 200,000 daltons, from about 2,000 daltons to about 200,000 daltons, from about 3,000 daltons to about 200,000 daltons, from about 4,000 daltons to about 150,000 daltons, from about 6,000 daltons to about 100,000 daltons, from about 2,000 daltons to about 50,000 daltons, from about 3,000 daltons to about 50,000 daltons, from about 8,000 daltons to about 50,000 daltons); and wherein the marker molecule has a known pI from about 0 to about 14, from about 2 to about 12, from about 3 to about 11, from about 4 to about 10, from about 5 to about 9, from about 6 to about 8. Segment A may be linked to Segment B in either orientation.

In one embodiment, Segment A may comprise 1-100 covalently linked amino acids (e.g., 1, 2, 3, 4, 5, 6, 10, 30, 50, 75, 100, etc. covalently linked amino acids or 10-30, 5-50, 15-40, 20-50, 30-60, 40-70, 50-80, 60-90, 70-100, etc. covalently linked amino acids), most preferably, 15 covalently linked amino acids. In a further embodiment, one, two or more (two, three, four, five, etc.) of the amino acids in Segment A are labeled. In another embodiment, one or more amino acids in Segment A are from tyrosine or tryptophan. In yet another embodiment, the labeled amino acid is a lysine. In yet another embodiment, the polypeptide or polynucleotide is labeled with carboxytetramethylrhodamine (TMR).

In another embodiment, Segment B may comprise from about 100 nucleotides (nt) to about 1,000 nt, from about 200 nt to about 2,000 nt, from about 300 nt to about 3,000 nt, from about 1,000 nt to about 5,000 nt, from about 3,000 nt to about 10,000 nt, from about 5,000 nt to about 20,000 nt, from about 6,000 nt to about 30,000 nt, from about 10,000 nt to about 50,000 nt, from about 20,000 nt to about 100,000 nt, from about 50,000 nt to about 200,000 nt, from about 70,000 nt to about 250,000 nt.

The invention further provides marker molecules having a molecular weight from about 300 daltons to about 3,000 daltons, from about 500 daltons to about 4,000 daltons, from about 1,000 daltons to about 5,000 daltons, from about 3,000 daltons to about 8,000 daltons, from about 5,000 daltons to about 12,000 daltons, from about 10,000 daltons to about 15,000 daltons, from about 12,000 daltons to about 18,000 daltons, from about 15,000 daltons to about 25,000 daltons, from about 20,000 daltons to about 30,000 daltons, from about 25,000 daltons to about 40,000 daltons, from about 30,000 daltons to about 50,000 daltons, from about 40,000 daltons to about 60,000 daltons, from about 50,000 daltons to about 80,000 daltons, from about 60,000 daltons to about 90,000 daltons, from about 75,000 daltons to about 110,000 daltons, from about 90,000 daltons to about 140,000 daltons, from about 110,000 daltons to about 160,000 daltons, from about 130,000 daltons to about 180,000 daltons, from about 140,000 daltons to about 200,000 daltons, from about 180,000 daltons to about 220,000 daltons, or from about 200,000 daltons to about 250,000 daltons.

The invention further provides marker molecules having a pI from about 0.5 to about 2, from about 1 to about 3, from about 2 to about 4, from about 3 to about 5, from about 4 to about 6, from about 5 to about 7, from about 6 to about 8, from about 7 to about 9, from about 8 to about 10, from about 9 to about 11, from about 10 to about 12, from about 11 to about 13, from about 12 to about 13.5, from about 2 to about 6, from about 3 to about 7, from about 5 to about 9, from about 6 to about 10, from about 8 to about 12, or from about 9 to about 13.

In another embodiment, the present invention relates to a marker molecule of wherein Segment A comprises a labeled organic molecule, L is a linker bond, and Segment B is a peptide, protein or polynucleotide, wherein Segment A can form bond L in only in one position of Segment B.

In a further embodiment, the present invention relates to a marker molecule wherein Segment A comprises a thioester and Segment B contains a single 1-amino-2-mercaptoethyl group. In yet another embodiment, the present invention relates to Segment A comprising a labeled polypeptide thioester or a labeled organic thioester. In a further embodiment, the present invention relates to Segment B comprising a protein, peptide or polynucleotide containing a 1-amino-2-mercaptoethyl group. In yet another embodiment, the present invention relates to the 1-amino-2-mercaptoethyl group in the protein or peptide comprising the N-terminal amino acid cysteine. In another embodiment, the present invention relates to the 1-amino-2-mercaptoethyl group in the polynucleotide comprising a single modified base. In yet another embodiment, the present invention relates to the peptide or protein comprising a recombinant protein constructed to have an N-terminal cysteine. In further embodiment, the present invention relates to the polynucleotide prepared with a single modified base by an enzymatic reaction. In another embodiment, the present invention relates to the marker molecule wherein Segment A comprises a single 1-amino-2-mercaptoethyl group and Segment B comprises a thioester. In another embodiment, the present invention relates to Segment A comprising a labeled polypeptide having the amino acid cysteine as the N-terminal amino group. In another embodiment, the present invention relates to Segment A comprising an organic molecule containing a 1-amino-2-mercaptoethyl group. In another embodiment, the present invention relates to Segment A comprising a cysteinyl carboxy ester or amide. In another embodiment, the present invention relates to Segment A constructed by automated peptide synthesis. In another embodiment, the present invention relates to the marker molecule wherein Segment A comprises an aldehyde reactive group and Segment B contains an aldehyde formed from oxidation of an N-terminal serine or threonine of a polypeptide or protein. In another embodiment, the present invention relates to marker molecule wherein Segment A comprises a labeled hydrazone. In another embodiment, the present invention relates to the marker molecule wherein L is a hydrazide bond.

In another embodiment, the present invention relates to a method of preparing a marker composition, the method comprising labeling an organic molecule and ligating it to a single position in a peptide, protein or polynucleotide. In another embodiment, the present invention relates to a method of labeling a marker molecule, comprising: ligating a first labeling molecule to a single position on a second molecule consisting of a protein, peptide or polynucleotide. In another embodiment, the present invention relates to a method of modifying the isoelectric point of a marker molecule comprising: ligating a first labeling molecule containing acidic or basic ionizable groups to a second molecule consisting of a protein, peptide or polynucleotide.

As used herein, the term “known pI,” when applied to marker molecules and their composition, means that the pI is theoretically calculated using the polynomial equations described in Sillero, A. et al., Analytical Biochem. 179:319-325 (1989) and Ribeiro, J. et al., Comput. Biol. Med. 20:235-242 (1990), which are incorporated herein by reference, or determined empirically.

In a further embodiment, the linker comprises a peptide bond or one of the following bifunctional linkers, without limitation:

—(CH₂)_{q}—NH—,
wherein q is 2-10;

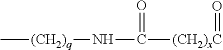

wherein q=2-5,
   x=2-12; and

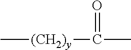

wherein y=1-3.

In one embodiment, Segment A may be preferably and specifically labeled with chromophores, fluorophores, or UV absorbing groups such as 5-carboxyfluoresceine (FAM), fluorescein, fluorescein isothiocyanate, 2′7′-dimethoxy-4′5′-dichloro-6-carboxyfluorescein (JOE), rhodamine, N,N,N′,N′-tetramethyl-6-carboxyrhodamine (TAMRA), tetramethyl rhodamine or carboxytetramethylrhodamine (TMR). In a further embodiment, Segment A may comprise a capture or binding tag such as biotin, fluoroscein, digoxigenin, polyhistidine or derivatives thereof. In another embodiment, Segment A may be used to modify the pI of Segment B by the presence of one or more acidic amino acids such as aspartate and glutamate or one or more basic amino acids such as lysine, arginine and histidine. In another embodiment, the addition of charged chromophoric groups or chromophores with a sulfonic acid also affect the pI. In a further embodiment, Segment A may be used to introduce reactive sites for covalent attachment of proteins.

In another embodiment, the present invention relates to the use of a labeled thioester wherein the labeled thioester may be a single amino acid thioester such as N-tetramethylrhodamine amide glycyl thioester to attach as a labeled Segment A to a protein, polypeptide, or polynucleotide having a 1-amino-2-mercaptoethyl group.

In a further embodiment, the present invention relates to the use of a labeled 1-amino-2-mercaptoethyl group to attach a label to a protein or polypeptide having a C-terminal thioester group.

In yet a further embodiment, the present invention relates to the use of labeled hydrazides and other aldehyde reactive groups as Segment A to attach a label to a protein or polypeptide having an oxidized (or oxidizable) N-terminal serine or threonine group.

Proteins may be modified so as to eliminate or introduce functional groups which may be targeted by selective reagents. For example, if a protein has no naturally occurring cysteines in its primary sequence, and nucleic acid (e.g., DNA) clone encoding the protein is available, mutagenesis may be undertaken to introduce one or more cysteines. Procedures for such modifications are well known in the art (Ausubel, F. M. et al., in Current Protocols in Molecular Biology, John Wiley and Sons, Chapter 8 (1995)). Briefly, in one example, the wild type nucleic acid encoding the protein to be modified is incorporated in a single stranded bacteriophage vector containing random uracil bases. The single stranded nucleic acid is hybridized with a complementary synthetic oligonucleotide sequence incorporating a codon at the site of modification encoding the new amino acid desired to be in that position. The new double stranded sequence is extended with T4 DNA polymerase and the resulting phage used to transform E. coli bacteria. The expressed protein may then be isolated by standard techniques well known to those of ordinary skill in the art.

Such procedures may be used not only to incorporate amino acids of interest, but also to replace amino acids and to eliminate reaction sites. For example, one may reduce the number of cysteine groups in a wild type protein so that there are few sites available for modification. Cysteine groups are particularly useful because of the large number of reagents available to selectively react with the sulfhydryl sidechain. Examples include maleimidyl or iodoacetamidyl derivatives of chromophoric compounds or other labels that are commercially available (e.g., eosin-5-maleimide, item E-118 from Molecular Probes, Inc., Bothell, Wash.; Oregon Green iodoacetamide, item 0-6010 also from Molecular Probes).

Other groups may also be selectively modified. For example, oxalyl groups on a labeling reagent will selectively react with the amidino group of arginine. So proteins may be cloned so as to add or delete arginines as described for cysteine. Such modified proteins may then be selectively labeled. As another example, N-hydroxysuccinimidyl esters will react with lysine groups on the protein. N-hydroxysuccinimidyl esters are also widely available commercially and include, for example, carboxyfluorescein-N-hydroxysuccinimidyl ester (available from Research Organics, Cleveland, Ohio, as item 1048C). Lysines may be selectively added or eliminated as desired using standard cloning techniques. Use of lysine or arginine as sites for modification is less attractive than cysteine, because there are generally more of these basic amino acids and their elimination often results in changes in the solubility characteristics and pI of the recombinant protein.

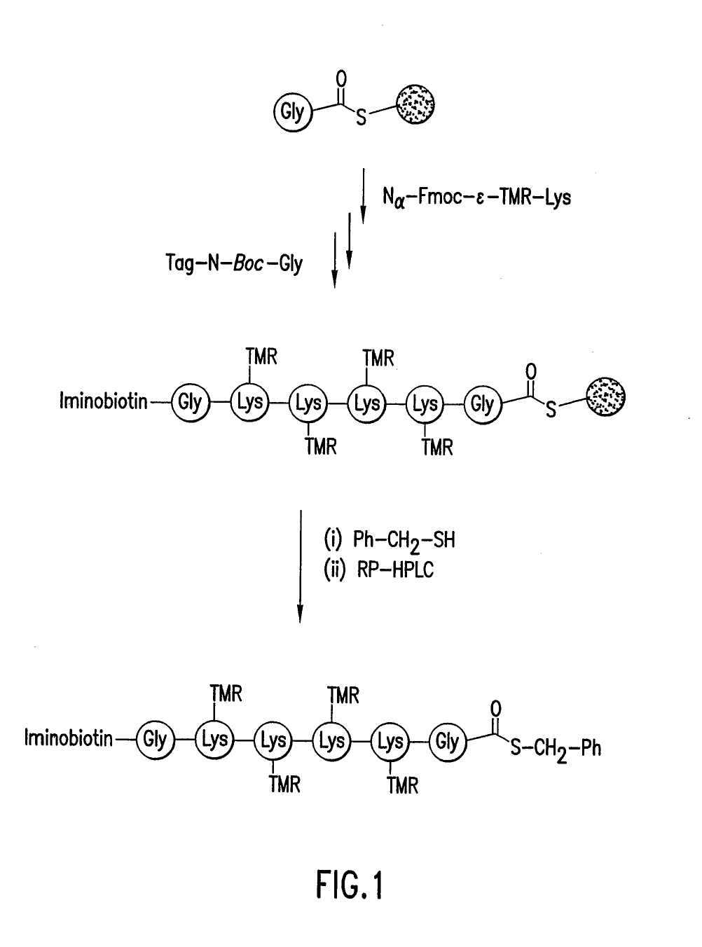

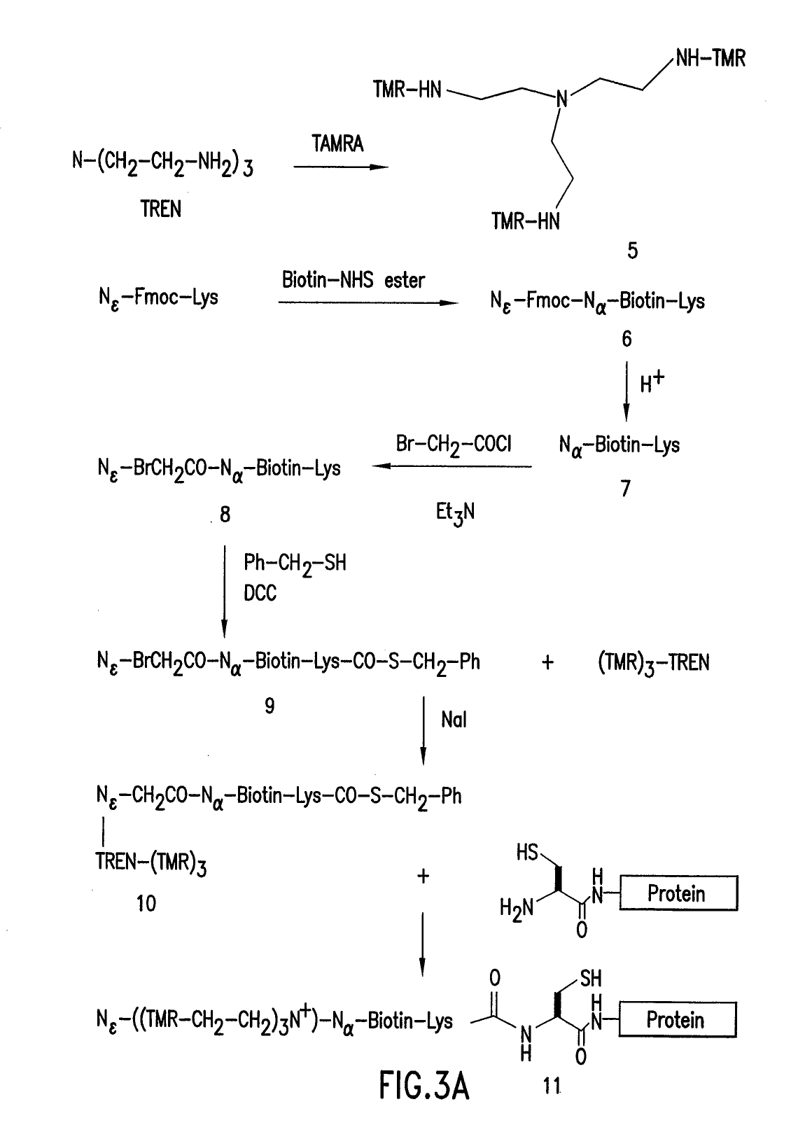

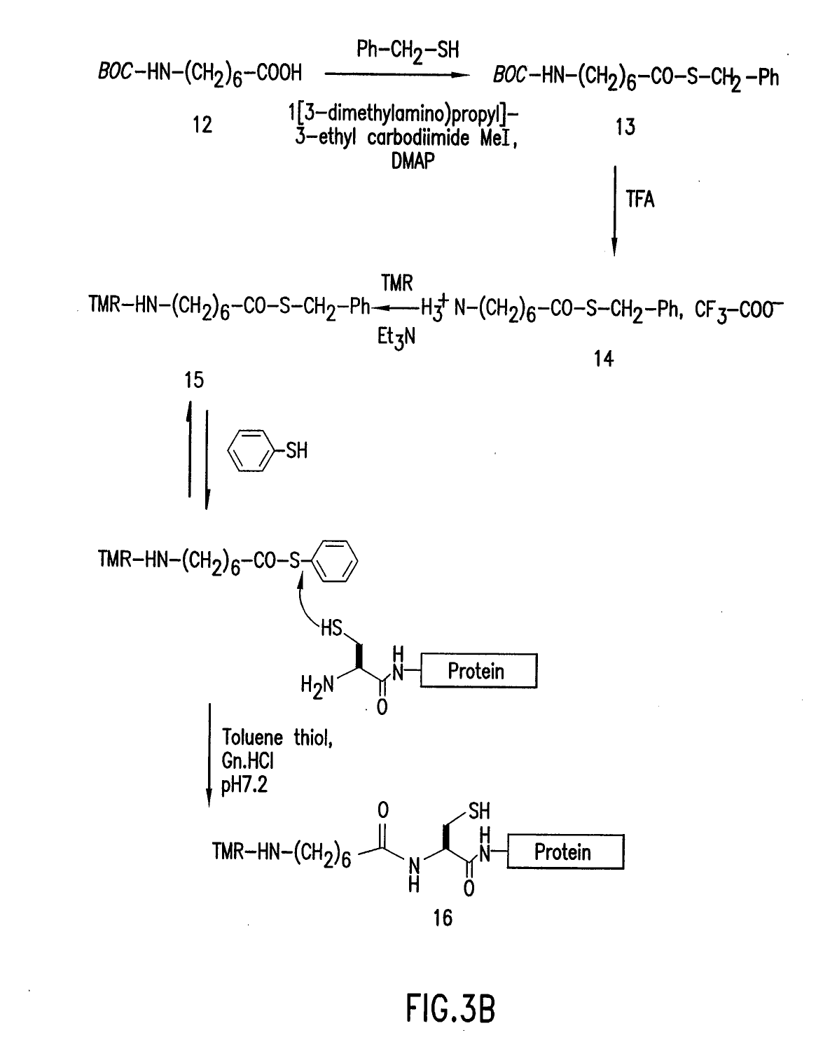

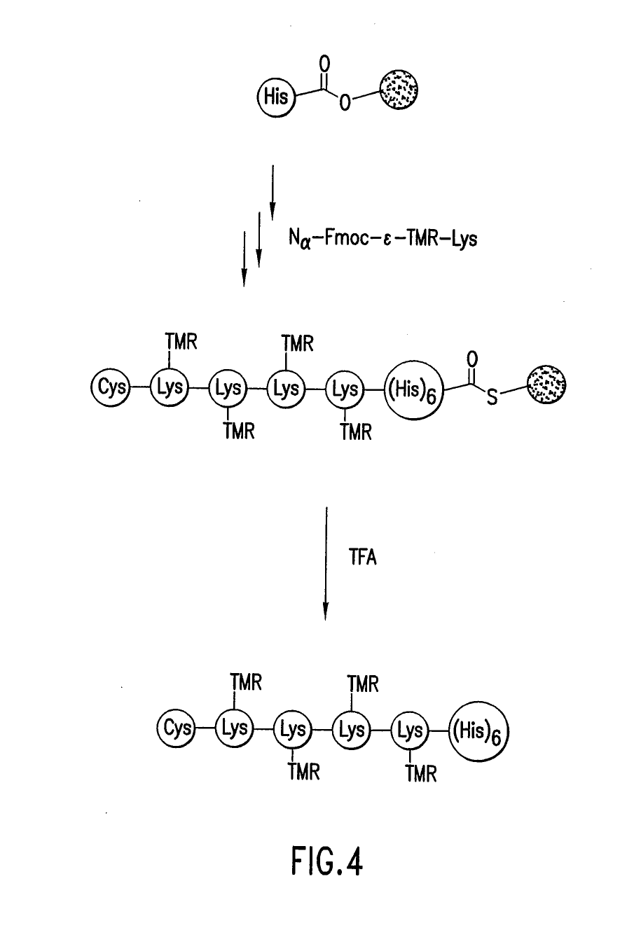

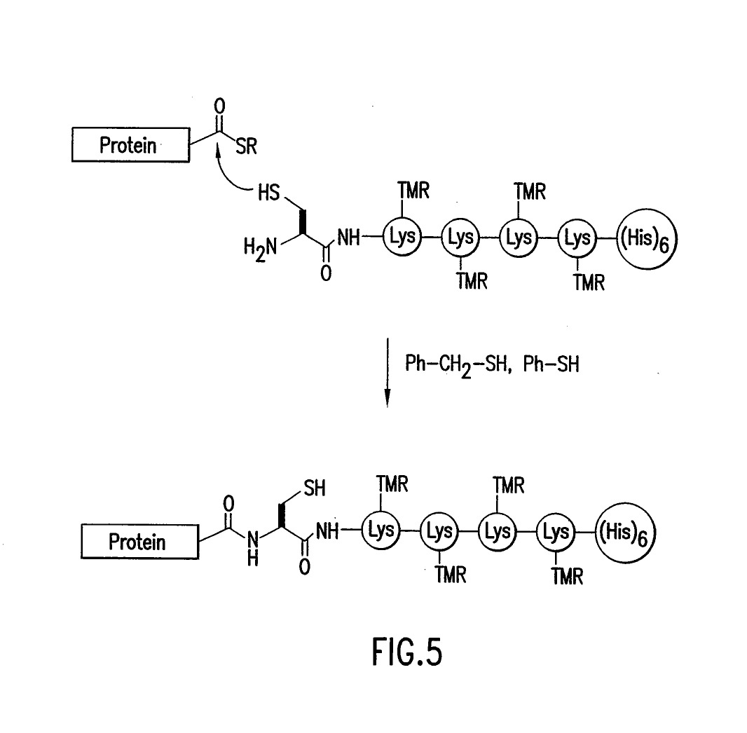

Nucleic acids may also be modified using the techniques described herein. For example, it is well known that modified bases such as biotin-16-dUTP, biotin-11-dUTP and biotin-14-dATP, among others, may be incorporated as labels by the action of polymerases when such building blocks are added to the typical nucleotide triphosphate mix used for in vitro synthesis of DNA (Ausubel, F. M. et al., in Current Protocols in Molecular Biology, John Wiley and Sons, 3.18.3 (1995)). Bases modified to contain 1-amino-2-mercaptoethyl groups may be prepared and incorporated by enzymatic action into DNA to form Segment B. Such labeling results in a nonspecific incorporation of the modified base into sites of the DNA. However, this group is reactive with molecules or macromolecules as Segment A bearing a thioester such as shown in FIGS. 1, 3A and 3B, so the reactive group could be used to attach labels to the nucleic acid after enzymatic synthesis. Molecules with a thioester may include polypeptides as well as smaller molecules.

As an example, N⁶-(6-aminohexyl)ATP is commercially available (Invitrogen Corporation). This compound may be readily ligated to a blocked cysteine activated with carbodiimide to form the 6-aminohexyl cysteinylamide. Once unblocked, this compound may be used in enzymatic synthesis of oligonucleotides as describe above. The resulting 1-amino-2-mercaptoethyl group is reactive with thioesters and allows the facile incorporation of labels and even the attachment of oligopeptides and proteins bearing a thioester group. Many other structural analogs of purine and pyrimidine bases may be modified in this manner, and as an example attachment to the N⁴ position of CTP or the N² position of guanine. Modified bases that are suitable for preparation of nucleotide triphosphates incorporating 1-amino-2-mercaptoethyl groups such as, without limitation, O4-Triazolyl-dT-CE (CE is β-cyanoethyl), 06-Phenyl-dI-CE, and O4-Triazolyl-dU-CE are also available from Glen Research, Sterling, Va., and from TriLink Biotechnologies, San Diego, Calif.

Another method of incorporating modified bases into a nucleic acid to form Segment B is to append it to the end of a nucleic acid chain. Terminal nucleotide transferase (Invitrogen Corporation) is a well known enzyme that may be used to append oligonucleotides to the 3′ end of DNA (Flickinger, J. et al., Nucleic Acids Res., 20:9 (1992)). This enzyme is used to incorporate biotinylated oligonucleotides and will readily incorporated bases modified with less bulky side groups such as 1-amino-2-mercaptoethyl groups capable of forming amide bonds with thioesters.

Yet another method of incorporation of labels into RNA employs guanylyltransferase (Invitrogen Corporation) which appends GMP onto the 5′ terminus of an RNA transcript which has a diphosphate or triphosphate group at the 5′ terminus. Use of a modified guanylyltriphosphate will give a base bearing a 1-amino-2-mercaptoethyl group that allows the incorporation of thioester-ligatable functions into RNA (Melton, D. A. et al., Nucleic Acids Res. 12:18 (1984)). Guanylyl transferase possesses GTP exchange properties so capped mRNA may be labeled with a thioester reactive base by incubating the capped mRNA with the enzyme and 1-amino-2-mercaptoethyl-modified GTP.

In particular embodiments, the present invention provides different chemical ligation strategies, further described below, to prepare homogeneous molecular marker compositions for gel electrophoresis systems.

As used herein, the term “isolated,” when applied to marker molecules, means that the molecules are separated from substantially all of the surrounding contaminants. “Surrounding contaminants” include molecules (e.g., amino acids, uncoupled Segment A, uncoupled Segment B, side products, etc.) associated with the production of the marker molecules but does not include molecules or agents associated with the isolation process or which confer particular properties upon either the marker molecules or compositions which contain the marker molecules. Examples of molecules which are typically not considered to be surrounding contaminants include water, salts, buffers, and reagents used in processes such as HPLC (e.g., acetonitrile). Thus, marker molecules which have been separated from unreacted molecules associated with marker molecule production by reverse phase HPLC (RP-HPLC), for example, are considered isolated even if present in a solution which contains 10% purification reagents such as organic solvents and buffers (e.g., acetonitrile and 10 mM Tris-HCl). This is the case even when the marker molecules are present in solutions at a concentration of, for example, 75 μg/ml. Further, the term “isolated” means that marker molecules being isolated are at least 90% pure, with respect to the amount of contaminants. In other words, the marker molecules which are isolated are separated from at least 90% of the surrounding contaminants.

The invention further includes isolated marker molecules, as well as compositions comprising one or more (e.g., one, two, three, four, five, six, eight, ten, twelve, twenty, fifty, etc.) isolated marker molecules, methods for preparing isolated marker molecules, methods for preparing compositions comprising isolated marker molecules, methods for using isolated marker molecules, and methods for using compositions comprising one or more (e.g., one, two, three, four, five, six, eight, ten, twelve, twenty, fifty, etc.) isolated marker molecules. The invention also includes compositions comprising one or more isolated marker molecules.

Marker molecules of the invention may be isolated and/or purified by any number of methods. Examples of such methods include HPLC (e.g., reverse phase HPLC), fast protein liquid chromatography (FPLC), cellulose acetate electrophoresis (CAE), isoelectric fractionation, column chromatography (e.g., affinity chromatography, molecular sieve chromatography, ion exchange chromatography, etc.), capillary zone electrophoresis, dialysis, isoelectric focusing, and field-flow fractionation.

One example of an apparatus which may be used to isolate and/or purify marker molecules of the invention is the Hoefer Isoprime isoelectric purification unit of Amersham Pharmacia Biotech Inc. (Piscataway, N.J. 08855) (Catalog No. 80-6081-90).

Chemical ligation involves a chemoselective reaction between synthetic unprotected oligopeptides, polynucleotides, organic compounds, macromolecules or small molecules, termed Segment A, with another unprotected protein (e.g., synthetic, recombinant or native proteins) or modified nucleic acid of known mass and charge, termed Segment B. The ligation reaction is site-specific and allows only a single specific coupling reaction between one site on one segment and one site of another segment, in the presence of other potentially reactive groups. Chemical ligation is useful for joining, for example, two segments which are both polypeptides. Peptides may be made by stepwise solid phase peptide synthesis and may have either an N-terminal cysteine (or N^{α}-(1-phenyl-2-mercaptoethyl)) or C-terminal thioester depending on the ligation strategy. Incorporation of chromophoric, acidic, and basic groups into the peptide chain may be achieved by using amino acids labeled with such groups during peptide synthesis.

Chemical ligation of proteins has the following advantages in the present invention:
It is site-specific and allows only a single specific coupling reaction between the C_{α} of one segment (e.g., Segment A or Segment B) and N_{α} of another segment (e.g., Segment A or Segment B), in the presence of other reactive groups.
It generates only one product.
The resulting product has a known pI and a known molecular weight. These parameters can be determined theoretically and experimentally.
It allows protein labeling using chromophores and fluorophores in a consistent, reproducible fashion.
It allows nucleic acid labeling using chromophores and fluorophores in a consistent, reproducible fashion.
It can be used to alter the pI of proteins. The incorporation of charged amino acid residues, or of charged chromophoric groups into Segment A, will alter the pI of the final protein product. For example, the guanidino group of arginine (pKa>12) will shift the pI of the product to basic pH, whereas, chromophores with a sulfonic acid group (pKa of 1.5) will shift the pI of the product to acidic pH. Other charged chromophores or charged amino acids will have similar effects.
It allows manipulation of the molecular weight of proteins. For example, a 30-residue oligopeptide (Segment A) increases the molecular weight of the protein (Segment B) by approximately 3.0 daltons (kD), depending on the amino acid sequence, upon ligation.
It allows incorporation of tags into proteins. Addition of tags such as biotin, fluorescein, digoxigenin, polyhistidine to the synthetic peptide followed by ligation of the peptide to the protein generates a tagged protein. This tagging strategy may be used to facilitate purification.
It allows ligation of polynucleotides to labeled oligopeptides in a consistent, reproducible fashion.

In the present invention, depending upon the N-terminal amino acid or the C-terminal carboxylate of the protein (Segment B), ligation strategies such as Native Chemical Ligation, in vitro chemical ligation or site-specific modification may be employed for attaching Segment A to Segment B.

In particular aspects, the present invention provides for: 1) synthesis of segments A and B, 2) ligation of Segment A to Segment B to form molecular markers, and/or 3) use of the molecular marker as molecular weight and isoelectric point markers.

Proteins with low numbers of post-translational modification sites or comprising low numbers of post-translational modifications are useful for preparing marker molecules suitable for use in methods and compositions of the invention and may be incorporated into such markers, for example, as Segment A and/or Segment B. For purposes of illustration, when a protein is deamidated at an asparagine or glutamine residue, in most instances, the molecular weight of the protein, and hence a marker molecule which comprises this protein, will change relatively little. However, the change in the isoelectric point of the protein will typically be relatively pronounced. In a situation where a marker molecule contains, for example, ten potential deamidation sites, a composition comprising this marker may contain a considerable number of different molecular species which vary based on whether deamidation has occurred at one or more of these ten sites. In other words, since deamidation may occur in individual marker molecules at one or more of these deamidation sites and deamidation at each site results in a change in charge, deamidation leads to the formation of a heterogeneous population of marker molecules.

Other post-translational modifications may substantially change both the charge and molecular weight of a protein. Ubiquitin, for example, is a seventy-six amino acid residue protein which is highly conserved among eukaryotes. Further, proteins are typically ubiquinated at lysine residues, and proteins may be poly-ubiquinated. Due to the size of ubiquitin, substantial changes in the molecular weight, as well as the charge, of a protein can occur upon ubiquination.

In one aspect, the present invention is directed to marker molecules, and methods for preparing such markers, which are relatively homogenous with respect to differences in post-translational modifications. As explained below, there are several ways to generate marker molecules which are relatively homogenous with respect to differences in post-translational modifications. For example, markers may be formed using proteins which comprise no, few, fewer or a reduced number of post-translational modification sites. In addition, markers may be formed using proteins which are produced and/or stored in such a manner as to result in no, few, fewer or a reduced number of post-translational modifications. The second option noted above may result, for example, from the use of proteins which (1) comprise no, few, fewer or a reduced number of post-translational modification sites, (2) are produced in systems which do not result in the introduction of post-translational modifications (e.g., the proteins may be synthetically produced), or (3) a combination of (1) and (2).

Eukaryotic and/or prokaryotic expression systems which are genetically altered, mutated, or modified so as to produce proteins which contain no, few, fewer or a reduced number of post-translational modifications or post-translational modification sites may be used to produce proteins which are used to prepare marker molecules of the invention. In particular instances, these expression systems may also comprise cells genetically programmed or altered to block regions of protein which are subject to post-translational modifications. In other particular instances, genetic alterations, mutations, and/or modifications can be designed or selected such that one or more post-translational modification system is either (1) rendered non-functional or (2) inducibly or constitutively repressed.

As used herein, the term “few,” when used in reference to post-translational modification sites or post-translational modifications refers to proteins wherein less than 50%, less than 40%, less than 30%, less than 20%, less than 10%, less than 5%, less than 2%, or less than 1% of the individual amino acids which make up the protein are either (1) potential post-translational modification sites or (2) are post-translationally modified. As one skilled in the art would recognize, proteins may be subject to a considerable number of types of post-translational modifications. Thus, the term “few” may be used in reference to a single type of post-translational modification site or post-translational modification or multiple types (e.g., two, three, four, five, six, seven, ten, etc.) of post-translational modification sites or post-translational modifications.

As used herein, the term “fewer,” when used in reference to post-translational modification sites or post-translational modifications refers to proteins which exhibit less than an expected average number of one or more different types of post-translational modification sites or post-translational modifications than would be expected for an average protein from the particular organism from which the protein was derived. For example, if the average ratio of potential deamidation sites (e.g., sites in proteins where glutamine or asparagine residues are found) in proteins from a particular organism is 1:15 (i.e., one deamidation sites/15 amino acid residues), then a protein from that organism which has the ratio of potential deamidation sites of 1:20 is said to have “fewer” deamidation sites. Thus, the term “fewer” is relative and requires reference to the organism from which the protein is derived. Further, the term “fewer” will typically be used to describe native proteins which are selected for use in marker molecules of the invention due to their amino acid sequences. Proteins used to prepare marker molecules of the invention may have any number of fewer post-translational modification sites or post translational modifications. In particular instances, proteins used to prepare marker molecules of the invention will have at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99% fewer of one or more post-translational modification sites or post translational modifications as compared to average proteins of the particular organism in which the protein is naturally found.

As used herein, the term “reduced number,” when used in reference to post-translational modification sites or post-translational modifications refers to proteins that have been altered to decrease the number of post-translational modification sites or post-translational modifications. One example of a situation where the number of post-translational modification sites is reduced is where a protein which contains three glutamine and one asparagine residue is modified to remove two of the glutamine residues. Thus, the number of deamidation sites is reduced by two. As above from the term “few”, the term “reduced” may be used in reference to a single type of post-translational modification site or post-translational modification or multiple types (e.g., two, three, four, five, six, seven, ten, etc.) of post-translational modification sites or post-translational modifications. In particular instances, proteins used to prepare marker molecules of the invention will exhibit a reduction in the number of one or more post-translational modification sites or post translational modifications of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%.

Known post-translational modifications include, but are not limited to, acetylation, acylation, ADP-ribosylation, amidation, carbamylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, deamidation, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Such post-translational modifications are well known to those of skill in the art. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (Methods of Enzymology 182:626-646 (1990)) and Rattan et al. (Ann. New York Acad. Sciences 663:48-62 (1992)). In a particular aspect, proteins used to prepare marker molecules of the present invention may contain no, few, fewer or a reduced number of post-translational modifications or sites for such post-translational modifications listed above, as well as other post-translational modifications. Thus proteins used to prepare marker molecules of the invention, as well as the marker molecules themselves, may comprise no, few, fewer or a reduced number of post-translational modifications or post-translational modification sites for one or more of the post-translational modification processes referred to above, or other post-translational modification processes.

A number of post-translational modifications occur in regions of proteins wherein more than one amino acid residue is required for modification recognition. In other words, while post-translational modifications generally occur at a single amino acid residue within a protein, intramolecular disulfide bonds being an exception, post-translational modifications often require a particular recognition sequence comprising more than one amino acid residue or a particular local conformation (e.g., secondary structure). The invention thus includes marker molecules which comprise proteins which contain no, few, fewer or a reduced number of such recognition sites, as well as methods for making and using such markers and compositions comprising such markers. Such marker molecules may be prepared, for example, using proteins where a single amino acid comprising a post-translational modification recognition region is altered or missing or where local conformation required for one or more particular post-translational modifications is lacking or is disrupted.

As noted above, proteins which may be used to prepare marker molecules of the invention include proteins which comprise no, few, fewer or a reduced number of post-translational modifications or post-translational modification sites. Examples of amino acids (e.g., L-amino acids) which are subject to post-translational modifications include the following: alanine, asparagine, aspartic acid, glutamine, glutamic acid, glycine, histidine, phenylalanine, proline, methionine, cysteine, lysine, tyrosine, serine, and threonine. The invention thus includes marker molecules which contain no, few, fewer or a reduced number of one or more (e.g., one, two, three, four, five, etc.) of the amino acid residues referred to above, as well as methods for making and using such markers and compositions comprising such markers. These proteins may comprise, for example, (1) repeating sequences of amino acid residues or repeating sets of two or more (e.g., two, three, four, five, six, etc.) amino acid residues, (2) amino acid sequences which are essentially random, or (3) predefined amino acid sequences which are not based on any particular order of the amino acids.

Amino acid residues which, with certain exceptions, are not subjected to post-translational modifications include glycine, methionine, tryptophan, alanine, valine, leucine, and isoleucine. Thus, the invention includes marker molecules in which Segment A and/or Segment B is a protein wherein at least 40%, at least 50%, at least 60%, at least 70%, at least 80%, at least 85%, at least 90%, at least 95% of the amino acid residues present are one or more (e.g., one, two, three, four, five six, or seven) of these amino acid residues set out directly above.

Examples of repeating sequences of amino acid residues or repeating sets of two or more (e.g., two, three, four, five, six, etc.) amino acid residues which, under particular circumstances, may be subject to no, few, fewer or a reduced number of post-translational modifications include proteins comprising poly-glycine (e.g., a protein comprising 25, 30, 40, or 50 glycine residues), repeating sets of glycine and alanine residues (e.g., a protein comprising 10, 15, 25, 30, 40, or 50 sets of Gly-Ala residues), repeating sets of glycine, alanine, and valine residues (e.g., a protein comprising 10, 15, 25, 30, 40, or 50 sets of Gly-Ala-Val residues), etc.

Amino acid residues such as glycine and alanine are subject to post-translational modifications such as GPI-anchoring (see Nalivaeva and Turner, Proteomics 1:35-747 (2001)). Thus, when these amino acid residues are present in a protein and post-translational modification at these residues is not desired, the protein may be produced in a manner such that GPI-anchoring does not occur.

The invention also includes marker molecules wherein Segment A and/or Segment B comprises a protein which does not contain any one, two, three, four, five, six, seven, eight, nine, ten, eleven, twelve, thirteen, fourteen, or fifteen of the following amino acids: alanine, arginine, asparagine, aspartic acid, glutamine, glutamic acid, glycine, histidine, phenylalanine, leucine, isoleucine, proline, methionine, cysteine, lysine, tryptophan, tyrosine, serine, valine, and threonine. These proteins will typically also have other characteristics referred to herein (e.g., will be of a particular molecular weight).

Marker molecules of the invention will typically have a predefined molecular weight and isoelectric point. Further, in order to function as a member of a set of markers which vary in both molecular weight and isoelectric point, typically it will be necessary to include amino acids which may be subject to particular post-translational modifications. As noted elsewhere herein, whether and/or when post-translational modification occurs, as well as the number of post-translational modifications present, can be regulated by the selection of the protein production method and/or the conformation of the protein where the amino acid residue which functions as the post-translational modification site is located. For example, ubiquination is believed to occur exclusively in eukaryotic cells. Thus, when a ubiquination site is present in a protein, ubiquination can be prevented by either synthetically producing the protein or by expressing the protein in a prokaryotic cell. Similar methods can be used, for example, to produce proteins which contain other post-translational modification sites (e.g., glycosylation sites) which are not post-translational modified at those sites (e.g., not glycosylated).

One example of a post-translational modification which is not typically directed by biological processes is deamidation. Thus, a purified protein, for example, which contains amino acid residues which can undergo deamidation (e.g., asparagine (N) and glutamine (Q) residues) will often undergo deamidation during storage. When a protein, or marker molecule which contains such a protein, contains more than one asparagine and/or glutamine residues and these amino acid residues undergo deamidation, the result is a heterogeneous population of molecules in which the degree of heterogeneity varies with the number of amino acid residues in each protein which are capable of undergoing deamidation and the differences between the individual molecules in which these amino acid residues have undergone deamidation. In other words, if all of the amino acid residues present in molecules which are capable of undergoing deamidation have either not undergone deamidation or have undergone deamidation, then a homogenous population of molecules will be present. However, if partial deamidation has occurred, the heterogeneity of the population will be determined by the degree to which deamidation of the individual amino acid residues in the molecules has occurred. Similar considerations apply to other forms of post-translational modifications (e.g., glycosylation, ubiquination, etc.).

In particular aspects, the marker molecules of the present invention, as well as proteins used to prepare marker molecules of the invention, may have no, few, fewer, or a reduced number of asparagine and/or glutamine residues within their sequences. The invention further includes methods for preparing and using such markers and compositions comprising such markers.

The invention further includes methods for selecting proteins to be used as Segment A and/or Segment B of marker molecules. These proteins may be selected by any method including visual review of (1) nucleotide sequences of coding regions or putative coding regions to identify nucleic acid molecules which encode proteins that contain no, few, fewer, or a reduced number of one or more post-translational modification sites or (2) amino acid sequences of proteins to identify proteins which contain no, few, fewer, or a reduced number of one or more post-translational modification sites. In addition, a computer program can be used to conduct the above review and identify proteins or nucleic acid molecules which encode such proteins suitable for preparing marker molecules. One example of a computer program which would identify proteins suitable for preparing marker molecules is a program which reviews amino acid sequence data by first identifying amino acid sequences data which correspond to individual full-length proteins or fragments thereof and then reviews the data to identify full-length proteins or fragments thereof which contain no, few, fewer, or a reduced number of one or more post-translational modification sites. Thus, the invention provides, in part, methods for selecting proteins for use in preparing marker molecules of the invention, as well as marker molecules prepared using proteins selected by these methods.

In one particular instance, the genome sequence for Eschericia coli strain K12, which is accessible through the National Library of Medicine GenBank database (www.ncbi.nlm.nih.gov) (see, e.g., sequence ID NC_{—}000913) was searched using a computer program for protein sequences containing no asparagine or glutamine residues, which are potential sites of deamidation that can lead protein heterogeneity in a protein population. A short PERL script (Practical Extraction Report Language) was used to identify (1) the longest amino acid sequences in the genome containing neither of these amino acids and (2) individual proteins which contained neither of these amino acids. Alternatively, amino acid sequences expressed in the 4,639,221 bp DNA may be searched manually (e.g., by visual inspection) for sequences which contain no, few, fewer, or a reduced number of sites susceptible to one or more post-translational modifications.

A number of proteins of Eschericia coli strain K12 proteins which contain no deamidation sites have been identified and may be suitable for preparing marker molecules of the invention. Examples of such proteins include the following:

(SEQ ID NO: 11)
MHTGSTTLPDFFAGMSDDFTPPIFAGYCRDDSHELRFRLYALL;
(SEQ ID NO: 12)
MKAIFVLKGWWRTS;
(SEQ ID NO: 13)
MSFMVSEEVTVKEGGPRMIVTGYSSGMVECRWYDGYGVKREAFHETELV
PGEGSRSAEEV;
(SEQ ID NO: 14)
MKHIPFFFAFFFTFP;
(SEQ ID NO: 15)
MTISDIIEIIVVCALIFFPLGYLARHSLRRIRDTLRLFFAKPRYVKPAG
TLRRTEKARATKK;
(SEQ ID NO: 16)
MTALLRVISLVVISVVVIIIPPCGAALGRGKA;
(SEQ ID NO: 17)
ALLWLTGSLWGRDWSFVKIAIPLMILFLPLSLSFCRDLDLLALGDARAT
TLGVSVPHTRFWALLLAVAMTSTGVAACGPISFIGLVVPHMMRSITGGR
HRRLLPVSALTGALLLVVADLLARIIHPPLELPVGVLTAIIGAPWFVWL
LVRMR;
and
(SEQ ID NO: 18)
GASLGEMIKEEMGPVPGTIALFGCFLIMIIILAVLALIVVKALAESPWG
VFTVCSTVPIALFMGIYMRFIRPGRVGEVSVIGIVLLVASIYFGGVIAH
DPYWGPALTFKDTTITFALIGYAFVSALLPVWLILAPRDYLATFLKIGV
IVGLALGIVVL.

In addition, when Segment A and/or Segment B is a protein, post-translational modifications may occur at either the carboxyl terminus or amino terminus Examples of post-translational modifications which occur at such termini include formylation, acetylation, pyroglutamate formation, GPI-anchoring, amidation, and polyglycylation. Alternatively, the amino and/or carboxyl termini may be blocked to prevent post-translational modifications or the proteins may be produced under conditions in which post-translational modification does not occur. Further, when Segment A and/or Segment B is a protein, these proteins may be produced under conditions in which one or both termini will not be post-translationally modified.

The invention further includes marker molecules which contain a particular numbers of sites (e.g., one, two, three, four, five, six, seven, eight, nine, ten, etc. sites) for the direct or indirect attachment of labels. These attachments sites may comprise charged or uncharged chemical groups. Further, the attachment of labels to these sites may or may not result in the alteration to the charge of the attachment site chemical group. For instance, Segment B may be a protein which contains one, two, three, four, five, six, seven, eight, nine, ten, etc. sites for the direct or indirect attachments of labels. As a specific example, a protein which is about 159 amino acids in length (e.g., the putative carbon starvation protein of E. coli) and contains two cysteine residues may be contacted with a label (e.g., a dye) under conditions which allow for direct (e.g., without the presence of an intervening linker) covalently attachment to the cysteine residues. Attachment of the dye to the cysteine residues, which are normally uncharged at neutral pH, will typically result in the formation of a heterogeneous population of molecules. More specifically, assuming both cysteine residues are accessible to the label but the labeling process does not go to completion, four groups of molecules will be present in the population. These groups will be composed of the following molecules: (1) molecules which contain no label, (2) molecules wherein the label is attached to the first cysteine residue, (3) molecules wherein the label is attached to the second cysteine residue, and (4) molecules wherein the label is attached to both cysteine residues. When the label is used to detect the presence of the marker molecules, the first group of molecules will not be detected. Further, the second and third groups of molecules will generally migrate virtually identically with respect to both molecular weight and isoelectric point. Finally, the fourth group will typically migrate differently and be distinguishable from the molecules in the second and third groups after separation by isoelectric point. Thus, when the above mixture, for example, (1) is separated on a two dimensional gel electrophoresis system in which molecular weight separation is used in the first dimensional and isoelectric point separation is used in the second dimension and (2) the label is used to detect the marker molecules, then two separate spots can be detected. In such an instance, the isoelectric points of the marker molecules will often be sufficiently close that when a relatively low resolution isoelectric focusing gel is used, either one spot or two spots which are located very close together will typically be detected but when a relatively high resolution isoelectric focusing gel is used two spots will be detected. Further, the isoelectric points of both marker molecules can be determined and/or calculated. Thus, the modification of a single protein by the direct or indirect attachment of a label can be used to generate a heterogeneous population of marker molecules in which a limited number of molecular species are present and the properties of these species, with respect their ability to function as marker molecules (e.g., molecular weight, isoelectric point, etc.), can be determined and/or calculated.

Thus, the invention includes, in part, methods for preparing marker molecules in which attachment of a label and/or post-translational modifications are used to generate populations of molecules wherein properties of the individual molecules present in the populations, with respect their ability to function as marker molecules (e.g., molecular weight, isoelectric point, etc.), can be determined and/or calculated. For example, such marker molecules may be prepared by providing a Segment A and/or Segment B molecule which has more than one (e.g., two, three, four, five, etc.) site (e.g., a site for label attachment, a post-translational modification site, etc.) which can be modified either by the attachment of a label or other molecule. As described above, when the process used to modify the Segment A and/or Segment B molecules does not go to completion, the number of species present in the mixed population will be determined by the number of modification sites present. The invention thus includes marker molecule populations which comprise two, three, four, five, six, seven, eight, nine, ten, etc. different marker molecules generated by the attachment of one or more label or other molecule to single starting molecule (e.g., a protein). The invention further includes methods for using such marker molecule populations and marker molecules, as well as the marker molecule populations and the marker molecules themselves and compositions comprising such marker molecule populations and marker molecules. These marker molecule populations are homogeneous in the sense that they are composed of a defined set of molecules, each of which has characteristics which can be readily determined either empirically or by calculation.

Further, when molecules are ligated to charged groups in a Segment B molecule, this ligation can be used to modulate the overall charge of the product (e.g., the Segment B molecule and/or the marker molecule). This charge modulation can occur, for example, in two different ways, as well as a combination of these two ways. First, if the group on the Segment B where attachment occurs is charged (e.g., positively or negatively charged), then the charge of the group may be altered (e.g., neutralized). Second, the molecule which is ligated to the Segment B molecule may contain its own charged groups and the addition of these charged groups to the product may confer a new isoelectric point upon the product. In many instances, charge modification by such methods will result in changes in isoelectric point of less than 1.0 pH unit, less than 0.8 pH units, less than 0.6 pH units, less than 0.4 pH units, or less than 0.2 pH units.

### Native Chemical Ligation

Native Chemical Ligation involves ligation of a macromolecule or small molecule containing a thioester (Segment A) with a protein (e.g., a native, recombinant or synthetic protein) having an N-terminal cysteine or an N^{α}-(1-phenyl-2-mercaptoethyl) group (Segment B). Recombinant proteins with desired termini may be produced in prokaryotic expression systems so that they have preferably no, few, fewer, or reduced numbers post-translational modifications. Marker molecules of the invention may be generated using native chemical ligation.

Native proteins are suitable as long as they have appropriate termini and have no, few, fewer, or a reduced number of sites susceptible to post-translational modification. Coupling of an auxiliary group, such as 1-phenyl-2-mercaptoethyl, to an N-terminal amino group is done post-transcriptionally when all active side chains are blocked.

Peptides suitable as Segment A, may be prepared by solid phase synthesis methods such as a highly optimized stepwise solid phase peptide synthesis (Kent, S. B. H., et al. U.S. Pat. No. 6,184,344 B1; Dawson, P. E., et al., Science 266:776-779 (1994); Lu, W., et al., J. Am. Chem. Soc. 118:8518-8523 (1996); Tolbert, T. J., et al., J. Am. Chem. Soc 122 (23):5421-5428 (2000); and Swinen, D. et al., Org. Lett. 2:2439-2442 (2000)).

Solid phase chemical synthesis is a technique for the systematic construction of a polypeptide from individual amino acids. Blocked amino acids (e.g., with α-amino groups) such as the following may be used in solid phase chemical synthesis: Alanine, Arginine, Aspartic Acid, Asparagine, Cysteine, Glutamic Acid, Glutamine, Glycine, Histidine, Iso-leucine, Leucine, Lysine, Methionine, Phenylalanine, Proline, Serine, Threonine, Tryptophan, Tyrosine and Valine. Amino acids other than the twenty amino acids commonly found in native proteins may also be incorporated into proteins by solid phase synthesis and may be used to prepare markers molecules of the invention. Examples of such non-natural amino acids include trans-4-hydroxyproline, 3-hydroxyproline, cis-4-fluoro-L-proline, dimethylarginine, homocysteine, the enantiomeric and racemic forms of 2-methylvaline, 2-methylalanine, (2-i-propyl)-β-alanine, phenylglycine, 4-methylphenylglycine, 4-isopropylphenylglycine, 3-bromophenylglycine, 4-bromophenylglycine, 4-chlorophenylglycine, 4-methoxyphenylglycine, 4-ethoxyphenylglycine, 4-hydroxyphenylglycine, 3-hydroxyphenylglycine, 3,4-dihydroxyphenylglycine, 3,5-dihydroxyphenylglycine, 2,5-dihydrophenylglycine, 2-fluorophenylglycine, 3-fluorophenylglycine, 4-fluorophenylglycine, 2,3-difluorophenylglycine, 2,4-difluorophenylglycine, 2,5-difluorophenylglycine, 2,6-difluorophenylglycine, 3,4-difluorophenylglycine, 3,5-difluorophenylglycine, 2-(trifluoromethyl)phenylglycine, 3-(trifluoromethyl)phenylglycine, 4-(trifluoromethyl)phenylglycine, 2-(2-thienyl)glycine, 2-(3-thienyl)glycine, 2-(2-furyl)glycine, 3-pyridylglycine, 4-fluorophenylalanine, 4-chlorophenylalanine, 2-bromophenylalanine, 3-bromophenylalanine, 4-bromophenylalanine, 2-naphthylalanine, 3-(2-quinoyl)alanine, 3-(9-anthracenyl)alanine, 2-amino-3-phenylbutanoic acid, 3-chlorophenylalanine, 3-(2-thienyl)alanine, 3-(3-thienyl)alanine, 3-phenylserine, 3-(2-pyridyl)serine, 3-(3-pyridyl)serine, 3-(4-pyridyl)serine, 3-(2-thienyl)serine, 3-(2-furyl)serine, 3-(2-thiazolyl)alanine, 3-(4-thiazolyl)alanine, 3-(1,2,4-triazol-1-yl)-alanine, 3-(1,2,4-triazol-3-yl)-alanine, hexafluorovaline, 4,4,4-trifluorovaline, 3-fluorovaline, 5,5,5-trifluoroleucine, 2-amino-4,4,4-trifluorobutyric acid, 3-chloroalanine, 3-fluoroalanine, 2-amino-3-fluorobutyric acid, 3-fluoronorleucine, 4,4,4-trifluorothreonine, L-allylglycine, tert-Leucine, propargylglycine, vinylglycine, S-methylcysteine, cyclopentylglycine, cyclohexylglycine, 3-hydroxynorvaline, 4-azaleucine, 3-hydroxyleucine, 2-amino-3-hydroxy-3-methylbutanoic acid, 4-thiaisoleucine, acivicin, ibotenic acid, quisqalic acid, 2-indanylglycine, 2-aminoisobutyric acid, 2-cyclobutyl-2-phenylglycine, 2-isopropyl-2-phenylglycine, 2-methylvaline, 2,2-diphenylglycine, 1-amino-1-cyclopropanecarboxylic acid, 1-amino-1-cyclopentanecarboxylic acid, 1-amino-1-cyclohexanecarboxylic acid, 3-amino-4,4,4-trifluorobutyric acid, 3-phenylisoserine, 3-amino-2-hydroxy-5-methylhexanoic acid, 3-amino-2-hydroxy-4-phenylbutyric acid, 3-amino-3-(4-bromophenyl)propionic acid, 3-amino-3-(4-chlorophenyl)propionic acid, 3-amino-3-(4-methoxyphenyl)propionic acid, 3-amino-3-(4-fluorophenyl)propionic acid, 3-amino-3-(2-fluorophenyl)propionic acid, 3-amino-3-(4-nitrophenyl)propionic acid, and 3-amino-3-(1-naphthyl)propionic acid. Thus, the invention includes marker molecules which contain one or more amino acids other than the twenty amino acids commonly found in proteins.

In solid phase chemical synthesis of peptides, amino acids are covalently linked one at a time to a polypeptide chain in a C-terminal to N-terminal direction. The C-terminal amino acid is generally coupled to a solid support, such as a cross-linked polystyrene resin or other suitable insoluble support. Typically, amino acids are systematically added, first to a resin linker, and then to the previously added amino acid. Each amino acid added to the growing chain must be chemically blocked at its α-amino group to prevent addition of numerous amino acids to the chain in a single cycle. Common blocking agents include tert-butyloxycarbonyl (BOC), 9-fluorenylmethoxycarbonyl (FMOC), acetamidomethyl, acetyl, adamantyloxy, benzoyl, benzyl, benzyloxy, benzyloxycarbonyl, benzyloxymethyl, 2-Bromobenzyloxycarbonyl, t-butoxy, t-butoxymethyl, t-butyl, t-butylthio, 2-chlorobenzyloxycarbonyl, cyclohexyloxy, 2,6-dichlorobenzyl, 4,4′-dimethoxybenzhydryl, 1-(4,4-dimethyl-2,6-dioxocyclohexylidene)ethyl, 2,4-dinitrophenyl, formyl, mesitylene-2-sulphonyl, 4-methoxybenzyl, 4-methoxy-2,3,6-trimethyl-benzenesulphonyl, 4-methoxytrityl, 4-methyltrityl, 3-nitro-2-pyridinesulphenyl, 2,2,5,7,8-pentamethylchroman-6-sulphonyl, tasyl, trifluoroacetyl, trimethylacetamidomethyl, trityl, xanthyl and others known to those of ordinary skill in the art. Such blocked amino acids are available from Sigma, St. Louis, Mo. Thus, each cycle of amino acid addition typically requires a deblocking step followed by an amino acid coupling step. Following the systematic coupling of select amino acids to form a polypeptide chain, the peptide may be released from the resin linker by the addition of an agent such as α-toluenethiol, or other suitable solvent. Further, the peptide may be recovered by purification techniques such as reverse phase, high-pressure liquid chromatography (RP-HPLC), affinity chromatography, or isoelectric fractionation.

In one example of the preparation of a suitable Segment A, the first amino acid is a glycine attached by thioesterification to a polystyrene bead and protected by an FMOC group. The building block amino acid is N_{α}-Fmoc-N_{ε}-TMR-Lysine, which is also blocked by FMOC, and can be obtained from many vendors, including Molecular Probes, (Eugene, Oreg., Catalogue No. F-11830). The blocking group is present to prevent unwanted reactions during the synthesis of the peptide. Extension of the peptide takes place by first removing the blocking group with an agent such as trifluoroacetic acid (TFA), and then allowing the newly free amino group to form a peptide bond with the next building block amino acid. Following extension of the resin linker, an N-terminal glycine may be added and labeled with iminobiotin, for recovery of the peptide, by treating the peptide with 2-iminobiotin-N-hydroxysuccinimide ester (available from Calbiochem-Novabiochem, San Diego, Calif., Catalogue No. 401778) in 0.1 M sodium phosphate as described by Greg T. Hermanson (in Bioconjugate Techniques, Academic Press, San Diego, Calif., p. 159 (1996)). After cleavage with α-toluenethiol, the crude thioester peptide may be purified by a process such as RP-HPLC (FIG. 1). Synthesis of Segment A by the above sequential and tightly controlled approach results in a homogeneous population of specifically labeled peptides. The methods of the present invention, such as those described above, may be used to sequentially introduce a predetermined number of charged and/or chromophoric groups into a sequence of amino acids to form a Segment A with a C-terminal thioester and may be readily carried out by one of ordinary skill in the art.

In another embodiment, Segment A may have the formula:

Cys-Yₙ—Z

where,
Y is one or more amino acid selected from the group consisting of alanine, arginine, aspartic acid, asparagine, cysteine, glutamic acid, glutamine, glycine, histidine, iso-leucine, leucine, lysine, methionine, phenylalanine, proline, serine, threonine, tryptophan, tyrosine and valine or non-natural amino acids such as trans-4-hydroxyproline, 3-hydroxyproline, cis-4-fluoro-L-proline, dimethylarginine, and homocysteine,
wherein at least one amino acid is labeled with a chromophore, fluorophore, or a UV absorbing group, preferably at least two amino acids are labeled;
Z is a C-terminal amino acid (C_{α}-carboxyl group may be modified to have an amide function); and
n=1-100 covalently linked amino acid, (e.g., 1, 2, 3, 4, 5, 6, 10, 30, 50, 75, 100, etc. covalently linked amino acids or 10-30, 5-50, 15-40, 20-50, 30-60, 40-70, 50-80, 60-90, 70-100 covalently linked amino acids) and/or 14 covalently linked amino acids. In another embodiment, Z may be any amino acid listed above including non-natural amino acids such as those set out herein. In another embodiment, the peptide is prepared via chemical synthesis, preferably solid phase chemical synthesis. In a further embodiment, the amino acid is labeled specifically with carboxytetramethylrhodamine (TMR). In yet a further embodiment, the labeled amino acid is lysine. In another embodiment, the N-terminal cysteine-labeled peptide may be ligated with a protein with known molecular weight having an α-thioester. Ligation occurs via Native Chemical Ligation or in vitro chemical ligation. In a further embodiment, the resulting product of the ligation reaction is a protein marker of known molecular weight and pI.

In a further embodiment, the present invention relates to a polypeptide, protein and marker molecules of the present invention further comprising a tag molecule. In another embodiment, the tag molecule is selected from the group consisting of biotin, fluorescein, digoxigenin, polyhistidine and their derivatives thereof. Tag molecules may be used to facilitate protein purification using ligands capable of binding to the tag such as avidin (binds to biotin), antibodies (binds to fluorescein or digoxigenin), lectin (binds to sugars), or chelated metal ions (bind to polyhistidine). In another embodiment, the polyhistidine comprises from two through ten contiguous histidine residues (e.g., two, three, four, five, six, seven, eight, nine, or ten contiguous histidine residues). The tag may also be a peptide tag comprising an amino acid sequence having the formula:

R₁-(His-X)ₙ—R₂,

wherein (His-X)ₙ represents a metal chelating peptide and n represents a number between two through ten (e.g., two, three, four, five, six, seven, eight, nine, or ten), and X is an amino acid selected from the group consisting of alanine, arginine, aspartic acid, asparagine, cysteine, glutamic acid, glutamine, glycine, histidine, iso-leucine, leucine, lysine, methionine, phenylalanine, proline, serine, threonine, tryptophan, tyrosine and valine. Further, R₂ is a polypeptide which is covalently linked to the metal chelating peptide and R₁ is either a hydrogen or one or more (e.g., one, two, three, four, five, six, seven, eight, nine, ten, twenty, thirty, fifty, sixty, etc.) amino acid residues. Tags of this nature are described in U.S. Pat. No. 5,594,115, the entire disclosure of which is incorporated herein by reference.

Segment B may be any N-terminal cysteine-containing protein (e.g., synthetic, recombinant or native), preferably of known molecular weight and pI. A recombinant protein with N-terminal cysteine may be prepared using any one of a number of E. coli expression vectors such as, but not limited to, pBAD/Thio-TOPO® (Invitrogen Corporation), pET (Invitrogen Corporation), pTWIN (New England Biolabs), pTYB (New England Biolabs), and others that are known in the art.

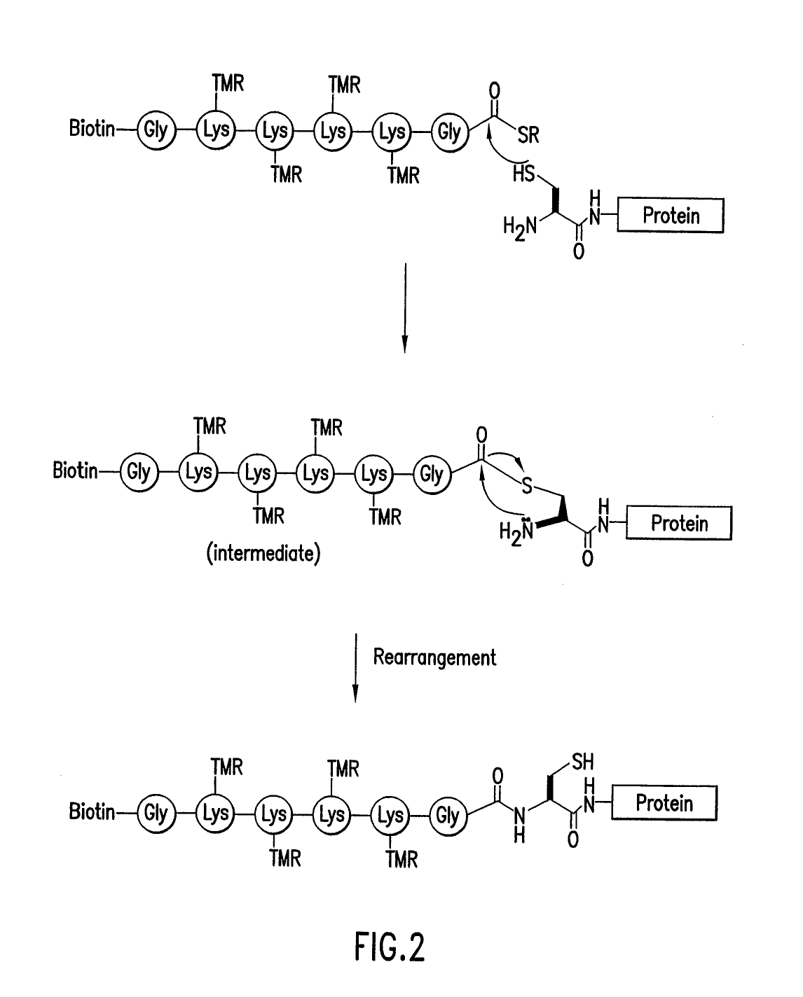

Ligation of Segment A to Segment B: The ligation reaction may be carried out according to the optimized protocol of Kent in U.S. Pat. No. 6,184,344 B1, the entire disclosure of which is incorporated herein by reference (FIG. 2).

The first step is a chemoselective reaction of the N-terminal cysteine of Segment B with the C-terminal thioester of Segment A (1.5 equivalents), for example, in 6M guanidine hydrochloride HCl, pH 7.5 in the presence of 1% toluenethiol and 5% thiophenol. Segment A's α-carbonyl thioester undergoes nucleophilic attack by the cysteine residue at Segment B's N-terminus, resulting in a thioester intermediate. The resulting thioester-linked intermediate undergoes spontaneous intramolecular acyl transfer to the nearby amine and forms a peptide bond (FIG. 2). The reaction is allowed to proceed to completion, e.g. in 24 hours, and the resulting product is purified, e.g. by affinity chromatography.

In another embodiment, Segment A may be a TMR-labeled organic thioester (see FIG. 3A). Acylation of triethylenetetramine (TREN, available from Aldrich, Milwaukee, Wis., Catalogue No. 90462) with 3.5 equiv. of an activated ester of carboxytetramethylrhodamine (TMR), available from Molecular Probes, OR (Catalogue No. e-6123), forms (TMR)₃-TREN 5. Acylation of N^{α}-Fmoc-Lysine with 2-iminobiotin-N-hydroxysuccinimide ester (Biotin-NS ester) yields N_{ε}-Fmoc-N_{α}-biotin-Lysine 6, see FIGS. 3A and 3B. Deblocking of the α-amino group of 6 followed by acylation with bromoacetyl chloride forms N_{ε}-bromoacetamido-N_{α}-biotinyl-Lysine 8. The carbodiimide coupling of 8 with α-toluenethiol results in 9. The alkylation of 5 with the thioester 9 in the presence of sodium iodide generates the quaternary ammonium salt 10 (Segment A) that upon coupling with Segment B under the same conditions described above affords 11 (chromophore to protein ratio=3).

In a further embodiment, Segment A may be a synthetic organic molecule that is labeled with a chromophore with a high extinction coefficient such as tetramethylrhodamine (TMR) as shown in FIG. 3B. In the reaction of N-Boc-8-heptanoic acid 12 with α-toluenethiol in the presence of 1-[(3-dimethylamino)propyl]-3-ethyl carbodiimide, methyl iodide and dimethylaminopyridine (DMAP, available from Aldrich, Milwaukee, Wis., Catalogue No. 33, 245-3) yields the corresponding thiobenzyl ester (FIG. 3B). Deprotection of the amino group of 13 in the presence of TFA and subsequent coupling of 14 to N-hydroxy succinimidyle ester of TMR generates the benzyl thioester derivative of N-TMR-8-heptanoic acid 15. The reaction of the thioester 15 (Segment A) with recombinant protein with N-terminal cysteine (Segment B) forms TMR-protein 16 (chromophore to protein ratio=1) that can be purified by dialysis.

In another embodiment, Segment B may have the formula:

Cysteine-oligonucleotide

Coupling of N^{α}-(6-aminohexyl)ATP to N-α-t-Boc-S-trityl-L-cysteine in the presence of a water soluble carbodiimide such as EDC forms N-α-t-Boc-S-trityl-6-aminohexylcysteinylamide. Deblocking of N-α-t-Boc-S-trityl-6-aminohexylcysteinylamide in the presence of trifluoroacetic acid and triisopropylsilane forms cysteine-ATP that can be added to an oligonecleotide chain enzymatically to generate cysteine-oligonucleotide (Segment B). Ligation of an oligopeptide with C^{α}-thioester labeled with chromophores, fluorophores, and UV absorbing groups to the cysteine-oligonucleotide segment in the presence of thiophenol and toluenethiol forms a labeled oligopeptide-oligonucleotide.

### In Vitro Chemical Ligation

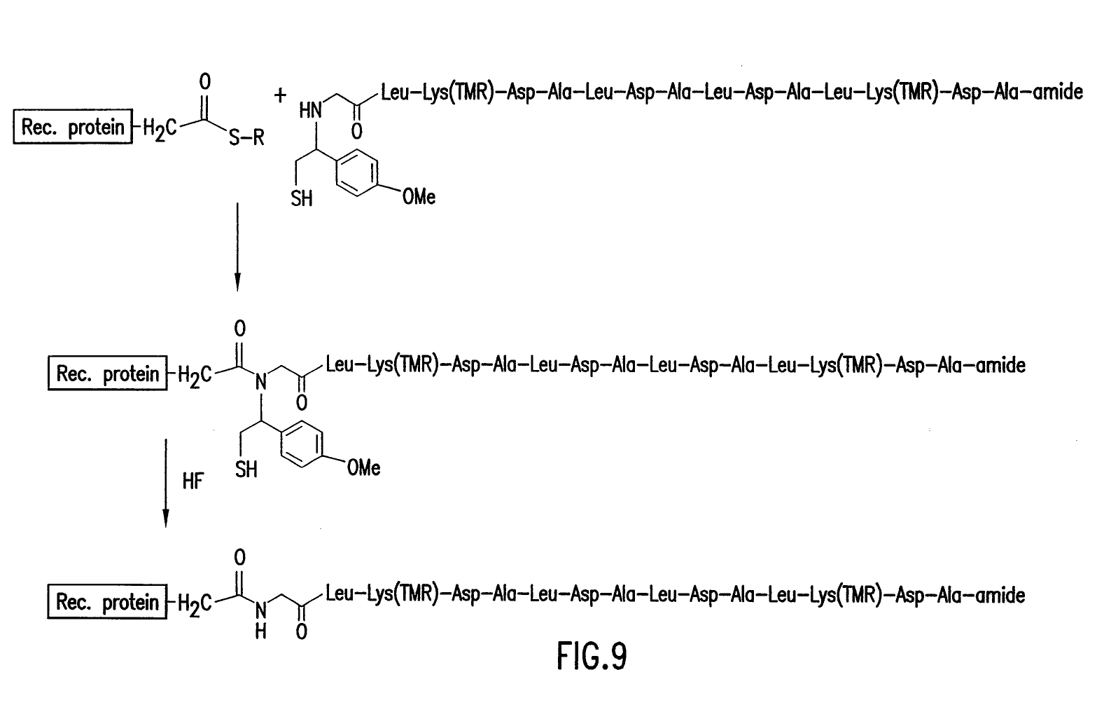

This method may involve ligation of Segment A, which is a labeled molecule with N^{α}-cysteine or N^{α}-(1-phenyl-2-mercaptoethyl) or small organic molecule which is labeled and contains 1-amino-2-mercaptoethyl moiety on a cysteine residue which is labeled through its carboxyl group to a recombinant protein with a C-terminal thioester (Evans, Jr., T. C., et al., J. Biol. Chem. 274:18359-8363 (1999)). However, the present invention is not limited to molecules with an N-terminal cysteines (Low, D. W., et al., Proc. Nat. Acad. Sci. U.S.A. 98:6554-6655 (2001)). Thus, a molecule which does not contain an N-terminal cysteine may be modified to form N_{α}-linked removable moiety (Canne, L. et al., J. Amer. Chem. Soc. 118:5891-5896 (1996)). In a specific embodiment, any synthetic peptide with a thiol-containing removable auxiliary moiety, such as 1-phenyl-2-mercaptoethyl, appended to the N-terminus, may be used as Segment A. Following the peptide bond formation, the auxiliary group can be removed in the presence of appropriate deblocking reagents. See FIG. 9. In another embodiment, any labeled organic molecule which contains 1-amino-2-mercaptoethyl group maybe be used as Segment A. In a specific embodiment, a labeled cysteine can be used as Segment A.

Segment B may be a protein (e.g., native, recombinant or synthetic protein) or a nucleic acid with a C-terminal thioester. In a further embodiment, the commercially available pTWIN1 expression plasmid such as IMPACT (New England Biolabs) with two modified mini inteins, Ssp DnaB and Mxe GyrA, may be employed to express Mxe GyrA intein genetically fused to the C-terminus of the protein of interest. Following affinity purification of the fusion protein (for example, via a chitin binding domain (CBD) placed downstream of Mxe GyrA), the target protein may be released simultaneously forming a thioester by treatment with an external thiol such as ethane thiol, n-butane thiol, or 2-mercaptoethanesulfonic acid (MESNA). Inteins and their use are described in U.S. Pat. No. 5,834,247, the entire disclosure of which is incorporated herein by reference. The IMPACT vectors have been used to express Maltose Binding Protein (MBP), McrB, T4 DNA ligase, Bst DNA polymerase Large Fragment, Bam HI, Bgl II, CDK2, CamK II and E. coli RNA polymerases with C-terminal thioester, as well as altered forms of these proteins.

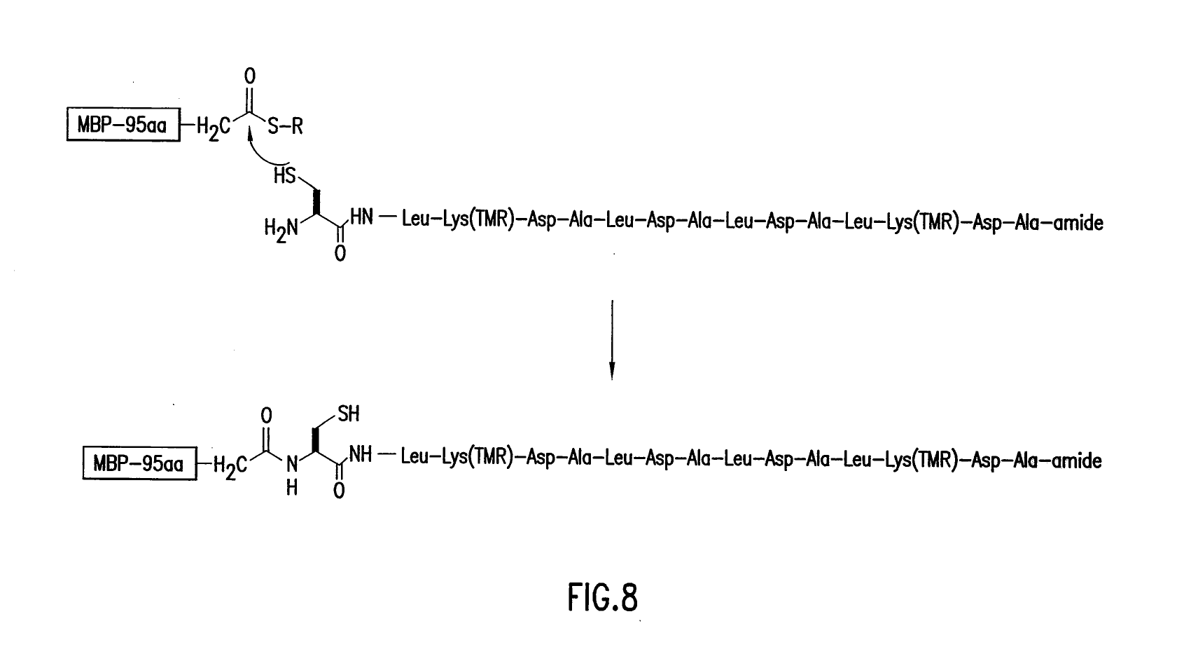

Ligation of Segment A to Segment B: The feasibility of in vitro chemical ligation to make visibly colored protein markers was first explored in a series of model reactions. A recombinant fragment corresponding to amino acids 1-92 of the 404 amino acid-long E. coli maltose binding protein (MBP) was genetically fused to the intein-CBD. The gene was modified at the DNA level to append the sequence Met-Arg-Met at the C-terminus. This addition was carried out to improve in vitro cleavage of the target protein (MBP-95aa) from intein as well as to enhance the ligation reaction. Exposure of the immobilized intein-fusion construct to MESNA has been shown to induce cleavage, and this was confirmed in the present system. The target protein was eluted as MBP-95aa-CO—S—CH₂—CH₂—SO₃Na and was characterized by mass spectroscopy (MS) and SDS gel. It was then evaluated whether the immobilized construct could be chemically ligated to a short synthetic peptide labeled with a chromophore (Cys-Lys(fluorescein)-Lys-Arg-Lys(fluorescein)-Lys-His-His-His-His-His-His) (SEQ ID NO:1) containing an N-terminal cysteine. Overnight exposure of the chitin beads to 1.0 mM of the peptide and 30 mM of MESNA at 4° C. generated MBP-107aa-(fluorescein)₂ which was characterized by mass spectrometry. MBP-95aa (10.6 kD, pI 5.12) was treated with Cys-Leu-Lys(TMR)-Asp-Ala-Leu-Asp-Ala-Leu-Asp-Ala-Leu-Lys(TMR)-Asp-Ala-amide (SEQ ID NO:3) in the presence of tributylphosphine, toluene thiol and thiophenol at room temperature, 37° C. and 50° C. (FIG. 8). The product was purified by RP-HPLC and characterized by MALDI/MS (13.0 kD, pI 4.75). In vitro chemical ligation using recombinant proteins has been reported (Muir, T. W. et al., Proc. Natl. Acad. Sci. USA 95:6704-6710 (1998)).

### Site-Specific Modification

Site-specific modification may involve conjugation of peptides or organic molecules to proteins with N-terminal serine or threonine. This method is described in Geoghegan, K. F. and Stroh, J. G., Bioconjugate Chem. 3:138-146 (1992).

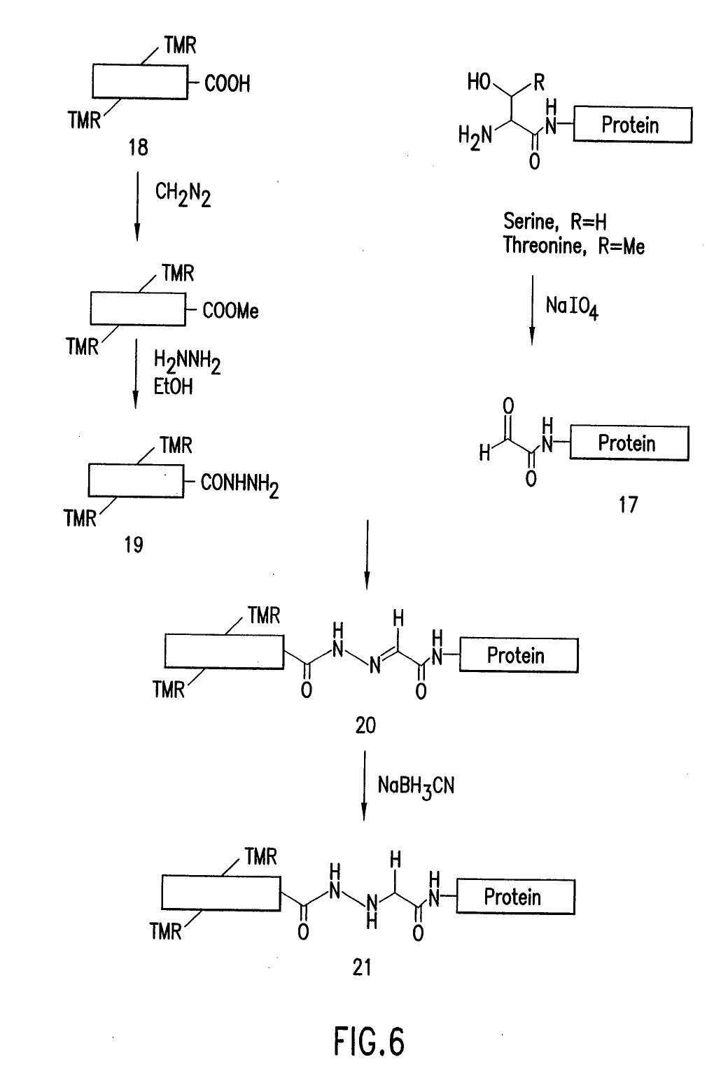

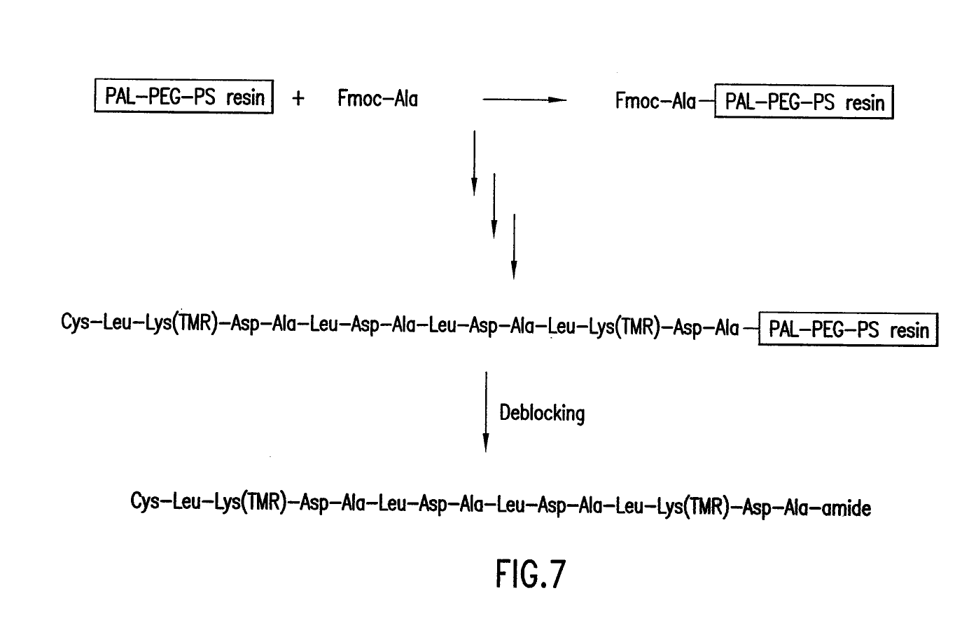

A further embodiment, depicted in FIG. 6, provides for the conjugation of peptides or organic molecules to proteins with N-terminal serine or threonine. The hydroxy group of these N-terminal amino acids is oxidized in the presence of periodate (available from Aldrich, Milwaukee, Wis.) to form an aldehyde, 17 (Segment B). Segment A is prepared from an oligopeptide or a synthetic organic molecule, such as 8-aminocaprylic acid, 7-aminoheptanoic acid and 6-aminohexanoic acid with a carboxyl function (18). Esterification of Cα of the peptide or carboxyl group of the organic molecule and subsequent exposure to hydrazine forms hydrazide 19. Coupling of Segment A with Segment B, e.g., using Geoghegan protocol (Geoghegan K. F. and Stroh, J. G., Bioconjugate Chem. 3:138-146 (1992)), forms the corresponding hydrazone 20 that can be reduced in the presence of sodium cyanoborohydride, to generate a more stable product, 21. Chromophoric labels can be introduced into Segment A during synthesis; therefore, the resulting product will be visibly colored. This procedure is less preferred than using either native peptide ligation or in vitro chemical ligation procedures because it requires the use of an oxidant to create the reactive group at the N-terminus that may damage the protein of Segment B.

The marker molecules and marker molecule compositions of the present invention may be used as standards in any system commonly used to separate macromolecules, e.g. by size, pI, or other physical or chemical property. The marker molecules and marker molecule compositions may be added to a matrix and exposed to an electromagnetic field which results in movement of the molecular markers through the matrix. Examples of such matrixes include, without limitation, agarose, cross-linked polyacrylamide gels, cross-linked dextran, DEAE-cellulose, DEAE-Sephadex, DEAE Sephacel and the like. The matrices may be in any form or shape, size or porosity. The shapes include slabs, blocks, tubes, columns, membranes and the like. The matrices may contain a number of additives which include, without limitation, denaturant, and buffers. In another embodiment, the marker molecules and marker molecule compositions may be used as markers in capillary electrophoresis. In another embodiment, the marker molecules and marker molecule compositions are used as standards when separating macromolecules by any other method including column chromatography, density gradient centrifugation, ion-exchange chromatography, size exclusion chromatography, thin layer chromatography, liquid chromatography, and the like.

In particular, marker molecules of the present invention may be used in gel electrophoresis systems such as those described below. A considerable number of gel electrophoresis separation systems are known in the art. Further, these systems operate to separate molecules by a variety of properties associated with the molecules being separated. Further, multiple separation principles may be combined to separate molecules (1) in a single gel electrophoresis system or (2) in different gels electrophoresis systems. In other words, molecules may be separated from each other in a one-dimensional gel system which separates molecules based on one or more (e.g., one, two, three, four, five, six, etc.) properties or the same molecules may be separated from each other using a two-dimensional gel, wherein each phase of the separation process separates molecules based on one or more (e.g., one, two, three, four, five, six, etc.) properties. Typically, when a two-dimensional gel system is used, molecules are separated in each of the two dimensions based on at least one different property (e.g., charge in the first dimension and molecular weight in the second dimension). Marker molecules of the present invention may be employed in one-dimensional and two-dimensional gel electrophoresis systems.

As noted above, gel electrophoresis systems may separate molecules based on a variety of properties. Examples of these properties including molecular weight, isoelectric point, and the ability of the molecules to bind detergents (e.g., non-ionic detergents), as well as combinations of these properties. Further, examples of gel electrophoresis systems in which marker molecules of the invention may be employed include SDS-polyacrylamide gel electrophoresis (SDS-PAGE), acid-urea gel electrophoresis, acid-urea gel electrophoresis conducted in the presence of one or more detergents (e.g., one or more non-ionic detergent such as TRITON X100™, sodium deoxycholate, NONIDET P40™, etc.), and isoelectric focusing. Markers molecules of the invention may be used, for example, with electrophoretic systems such as one-dimensional gel electrophoresis systems, two-dimensional gel electrophoresis systems, capillary electrophoresis systems, and electrokinetic chromatography systems, as well as other gel electrophoresis systems.

In one aspect, the invention includes marker molecules of uniform molecule weight, as well as compositions containing one or more (e.g., one, two, three, four, five, six, eight, ten, twelve, twenty, fifty, etc.) marker molecules which differ in molecular weight. These marker molecules are particularly suited for use with gel electrophoresis systems which separate molecules on the basis of molecular weight. Examples of gel electrophoresis systems which separate molecules mainly on the basis of molecular weight include SDS-PAGE systems (Laemmli, U.K., Nature 227:680-685 (1970)).

In another aspect, the invention includes marker molecules of uniform isoelectric point, as well as compositions containing one or more (e.g., one, two, three, four, five, six, eight, ten, twelve, twenty, fifty, etc.) marker molecules which differ in isoelectric point. These marker molecules are particularly suited for use with gel electrophoresis systems which separate molecules on the basis of isoelectric point (e.g., isoelectric focusing systems).

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Reaction of Cys-Ser-Thr-Met-Met-Ser-Arg-Ser-His-Lys-Thr-Arg-Ser-His-His-Val-OH (SEQ ID NO:2) with TMR-thioester 15 using Native Chemical Ligation

The model peptide, Cys-Ser-Thr-Met-Met-Ser-Arg-Ser-His-Lys-Thr-Arg-Ser-His-His-Val-OH (SEQ ID NO:2), was prepared by optimized stepwise solid phase peptide synthesis. The thioester 15 was prepared as outlined in FIG. 3B. To a 1 mL solution of 6.0 M guanidine hydrochloride buffered at pH 7.3 with 0.1 M sodium phosphate containing 5.0 mg (2.65×10-36 mmol) of the peptide was added 3.0 mg (1.5×10⁻³ mmol) of TMR-thioester 15 dissolved in 20 μL of acetonitrile. To this was added 10 μL (1%, v/v) toluenethiol and 30 μL (3%, v/v) thiophenol and stirred at room temperature under Argon overnight. Mass spectroscopy data and SDS gel electrophoresis showed that the product, TMR-labeled peptide was formed.

### Example 2

Cloning of Maltose Binding Protein-95aa (MBP-95aa) Gene into pTWIN1 Vector

TOPO Cloning of MBP-95aa Gene: Two restriction sites, Spe1 and Nde1, were introduced on either side of MBP-95aa gene. The PCR amplified gene was purified and TOPO-cloned into pCR-TOPO vector. The pCR-TOPOMBP-95aa gene was transformed into TOP10 competent cells and grew on LB/AMP plate overnight. Ten colonies were taken and used to inoculate ten 2-mL LB/AMP cultures (one colony/tube) and grown at 37° C. overnight. The DNA from each culture was isolated using S.N.A.P.™ (Simple Nucleic Acid Prep) Miniprep kit (Invitrogen Corporation, Carlsbad, Calif.) and analyzed by DNA sequencing.

Restriction Digestion and Ligation: The pCR-TOPOMBP-95aa was digested simultaneously with SpeI and NdeI at 37° C. overnight. The pTWIN1 vector was digested with the same enzymes. Both reaction mixtures were purified on a 1.2% agarose gel. The insertion of MBP-95aa gene into pTWIN1 plasmid was conducted at 14° C. for 3½ hours.

Transformation: TOP10 cells were transformed with the above ligation mixture and plated on LB/AMP/XgaI along with control experiments. Several 2-mL LB/AMP cultures were inoculated with different colonies (one colony/tube) and grew at 37° C. overnight. pTWIN1 MBP-95aa was isolated by S.N.A.P. Miniprep.

Screening for Insert: To confirm the insertion, pTWIN1 MBP-95aa was digested with SpeI and NdeI enzymes. This reaction resulted in two fragments: the insert, 250-300 bp and the backbone, ˜7000 bp.

### Cell Culture and Fusion Protein Expression

BL21/BAD cells were transformed with pTWIN1 MBP-95aa and were plated on LB/AMP and grew at 37° C. overnight. A 2-mL LB/CAR (200 μg carbenicillin/mL LB) culture was inoculated with one colony and grew at 37° C. overnight. 1 liter LB/CAR medium containing 0.01% glucose was inoculated with the above culture and grew at 30° C. Mid-log phase cells were induced with 0.1 mM isopropyl-1-β-D-galactopyranoside (IPTG) and 0.1% arabinose at 30° C. for 2½ hours.

### Cell Harvest

The cells from the induced culture were spun down at 5000×g for 15 minutes at 4° C. and the supernatant was discarded. At this stage, the cell pellets were stored at −80° C.

### Affinity Purification and On-column Cleavage

### Preparation of Crude Cell Extract

A 2.0 g pellet was resuspended in 100 mL of ice-cold lysis buffer (25 mM Tris pH 8.0, 800 mM KCl, 0.1 mM EDTA, 0.5% Triton X-100, 1.0 mM PMSF) and was split into two portions. Each portion was sonicated for 1 min×4. Combined lysate was clarified by centrifugation at 12000×g for 30 minutes at 4° C.

### Preparation of Chitin Column

A column packed with 15 mL of chitin beads (bed volume) was prepared and equilibrated with 100 mL of column buffer (20 mM Tris, pH 8.5, 500 mM NaCl, 0.1 mM EDTA, 0.1% Triton X-100.

### Loading the Clarified Cell Lysate

The clarified cell lysate was loaded onto the chitin column at a flow rate of 0.5 mL/min. The flow-through was collected and loaded onto the same column at a flow rate of 1.0-2.0 mL/min.

### Washing the Chitin Column

The column was washed with 500 mL of column buffer at a flow rate of 2.0 mL/min.

All traces of crude extract were washed off the sides of the column.

Induction of on-Column Cleavage

The column was loaded with 50 mL of MESNA buffer (200 mM mercaptoethane sulfonic acid in the column buffer), flushed quickly until the buffer is slightly above the chitin beads. The flow was stopped and the column was slowly rocked at room temperature overnight.

### Elusion of the Target Protein

Following on-column cleavage of the intein, MESNA derivative of MBP-95aa was released as α-thioester and eluted using column buffer. All fractions were analyzed by SDS-PAGE. Combined fractions were concentrated using Millipore Ultrafree—15 Centrifugal Filter Device Biomax—5K to yield 5.6 mg of the desired protein.

### Example 3

### Synthesis of Peptides

A peptide suitable as a “Segment A” and having the following amino acid sequence: Cys-Leu-Lys(TMR)-Asp-Ala-Leu-Asp-Ala-Leu-Asp-Ala-Leu-Lys(TMR)-Asp-Ala-amide (SEQ ID NO:3), was prepared by highly optimized stepwise solid phase peptide synthesis. In a 30-mL reaction vessel fitted with a glass frit 909 mg (0.2 mmol) of Fmoc-PAL-PEG-PS resin (Applied Biosystems, 0.22 meq.) was soaked in 10 ml of 20% of piperidine/DMF solution containing 0.05 M HOBt for 5 minutes. The liquid was drained, and the same procedure was repeated 2 more times. The resin was washed with 10 ml of DMF six times. In another reaction vessel, the carboxyl group of Fmoc-Ala (249.0 mg, 0.8 mmol) was activated with of 303.0 mg (0.8 mmol) O-benzotriazol-1-yl-N,N,N′,N′-tetramethyluronium hexafluorophosphate (HBTU) in the presence of 30.0 mg (0.2 mmol) of 1-hydroxybenzotriazole (HOBT) and 280.0 μL (1.6 mmol) of N,N-diisopropylethylamine (DIEA) in 10 ml of DMF. The mixture was stirred for 3 minutes at room temperature, added to the resin and stirred at room temperature for 1.5 hours. The mixture was washed with DMF several times. The activation and coupling of the second amino acid, Fmoc-Asp(O-t-Bu), was done under the same conditions described for Fmoc-Ala. The third amino acid, Fmoc-Lys(TMR) was purchased as N-hydroxysuccinimido ester (Molecular Probes). It did not require further activation and was added to the reaction mixture (250 mg 0.32 mmol), protected from light and left at room temperature overnight. Following Fmoc-Lys(TMR) coupling, the mixture was transferred into Applied Biosystems Pioneer Peptide Synthesizer vessel. A peptide having the amino acid sequence: Asp-Ala-Leu-Asp-Ala-Leu-Asp-Ala-Leu (SEQ ID NO:4), was then assembled onto the Lys(TMR)-Asp-Ala-resin. The synthesis protocol for the synthesizer was: 5 min deprotection step with piperidine/DMF (1:4, v/v) containing 0.05M HOBt, 1 hr coupling time with Fmoc-amino acid/HBTU/HOBT/DIEA (4:4:1:8). After the synthesis was done on the synthesizer, the reaction mixture containing Asp-Ala-Leu-Asp-Ala-Leu-Asp-Ala-Leu-Lys(TMR)-Asp-Ala-resin (SEQ ID NO:5) was transferred into the manual reaction vessel, and the rest of the sequence Cys-Leu-Lys(TMR)) was coupled stepwise and manually as described before (FIG. 8).

Deblocking: A reaction mixture containing 1.364 g of Cys-Leu-Lys(TMR)-Asp-Ala-Leu-Asp-Ala-Leu-Asp-Ala-Leu-Lys(TMR)-Asp-Ala-resin (SEQ ID NO:3) was added with 300 μL of scavenger mixture (thioanisole 10 ml/triisopropylsiline 4 ml/phenol 600 mg), 200 μl of mercaptopropionic acid (MPA) and 10 ml of 95% TFA/5% H₂O was left at room temperature for 3 hours with occasional stirring. A 100 ml of tert-butyl methyl ether (MTBE)/hexane (1:1) was added to the reaction mixture and centrifuged. The supernatant was decanted, and the residue was washed with 50 ml of MTBE/hexane (1:1) and centrifuged again. The solid was separated by decantation, extracted with 50 ml of 50% of acetonitrile in H₂O and lyophilized. The crude mixture was purified on preparative C-18 RP-HPLC to yield 198 mg of pure peptide that was MS analyzed by MS (Found 2397.67, Calc. 2398.71).

The following peptides were prepared:

(SEQ ID NO: 6)
Cys-Asp-Asp-Lys(TMR)-Asp-Asp-Asp-Asp-Leu-Ala-Asp-
Asp-Asp-Lys(TMR)-Asp-amide
(SEQ ID NO: 7)
Cys-Asp-Lys(TMR)-Asp-Ala-Asp-Asp-Leu-Ala-Asp-Leu-
Asp-Lys(TMR)-Asp-Ala-amide
(SEQ ID NO: 8)
Cys-Gly-Lys(TMR)-Ser-Gly-Ser-Gly-Lys-Ser-Gly-Lys-
Gly-Lys(TMR)-Ser-Gly-amide
(SEQ ID NO: 9)
Cys-Ala-Lys(TMR)-Leu-Lys-Ala-Lys-Ala-Lys-Leu-Ala-
Lys-Lys(TMR)-Leu-Ala-amide
(SEQ ID NO: 10)
Cys-Lys-Lys(TMR)-Lys-Ala-Lys-Leu-Lys-Ala-Lys-Lys-
Lys-Lys-Lys(TMR)-Ala-amide

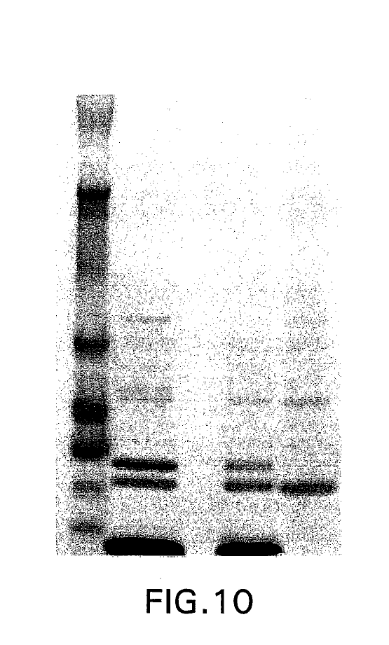

Ligation of Cys-Leu-Lys(TMR)-Asp-Ala-Leu-Asp-Ala-Leu-Asp-Ala-Leu-Lys(TMR)-Asp-Ala-amide (Segment A) (SEQ ID NO:3) to MBP-95aa (Segment B): A mixture of MBP-95aa (0.4×10⁻⁶ mmol, 4.0 mg) and Cys-Leu-Lys(TMR)-Asp-Ala-Leu-Asp-Ala-Leu-Asp-Ala-Leu-Lys(TMR)-Asp-Ala-amide (0.4×10⁻⁵ mmol, 8.9 mg) (SEQ ID NO:3) was stirred in 6.0 M guanidine hydrochloride buffered at pH 7.3 with 0.1 M sodium phosphate in the presence of 5 mM tri-butylphosphine (25 μL of 200 mM solution in 1-methyl-2-pyrrolidinone) and 20 mM mercaptoethanol. To this was added 3% (v/v) thiophenol as a catalyst and stirred at room temperature for 96 hours. Every 24 hours, 25 μL of 200 mM solution of tributylphosphine was added to the reaction mixture. The reaction mixture was monitored by SDS gel electrophoresis and it went to 60% completion. The desired product, MBP-110aa-(TMR)₂, was purified on preparative RP HPLC and characterized by SDS-gel and MALDI-MS (Found 13061.1, Calc. 13037.01; pI value 4.75). MBP-110aa-(TMR)₂, pI 4.75 was tested on NuPAGE Bis-Tris, 4-12% (Invitrogen Corporation) and 16% Tricine gel (Invitrogen Corporation) using MultiMark (Invitrogen Corporation) as protein marker; gel shown in FIG. 10.

The ligation of Cys-Asp-Asp-Lys(TMR)-Asp-Asp-Asp-Asp-Leu-Ala-Asp-Asp-Asp-Lys(TMR)-Asp-amide (SEQ ID NO:6) to MBP-95aa, results in a marker molecule, MBP(110a)-(TMR)₂; calculated pI 4.3. The ligation of Cys-Asp-Lys(TMR)-Asp-Ala-Asp-Asp-Leu-Ala-Asp-Leu-Asp-Lys(TMR)-Asp-Ala-amide (SEQ ID NO:7) to MBP-95aa results in a marker molecule, MBP(110a)-(TMR)₂; calculated pI 4.5. The ligation of Cys-Gly-Lys(TMR)-Ser-Gly-Ser-Gly-Lys-Ser-Gly-Lys-Gly-Lys(TMR)-Ser-Gly-amide (SEQ ID NO:8) to MBP-95aa results in a marker molecule, MBP(110a)-(TMR)₂; calculated pI 6.5. The ligation of Cys-Ala-Lys(TMR)-Leu-Lys-Ala-Lys-Ala-Lys-Leu-Ala-Lys-Lys(TMR)-Leu-Ala-amide (SEQ ID NO:9) to MBP-95aa results in a marker molecule, MBP(110a)-(TMR)₂; calculated pI 7.4. The ligation of Cys-Lys-Lys(TMR)-Lys-Ala-Lys-Leu-Lys-Ala-Lys-Lys-Lys-Lys-Lys(TMR)-Ala-amide (SEQ ID NO:10) to MBP-95aa results in MBP(110a)-(TMR)₂; calculated pI 9.5.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations, and other parameters without affecting the scope of the invention or any specific embodiment thereof, and that such modifications of changes are intended to be encompassed within the scope of the appended claims.

All publications and patents mentioned in this specification are indicative of the level of skill of those skilled in the art to which this invention pertains, and are herein incorporated by reference to the same extent as if each individual publication or patent was specifically and individually indicated to be incorporated by reference.

1-38. (canceled)

39. A marker molecule of the formula I:
Segment A-L-Segment B
wherein,
Segment A is a labeled molecule;
L is a linker or a bond; and
Segment B is a protein which contains no deamidation sites.

40. The marker molecule of claim 39, wherein Segment B is a protein which contains no arginine or glutamine residues.

41. The marker molecule of claim 40, wherein the proteins comprises an amino acid sequence selected from the group consisting of:
(a) the amino acid sequences shown in SEQ ID NO:11;
(b) the amino acid sequences shown in SEQ ID NO:12;
(c) the amino acid sequences shown in SEQ ID NO:13;
(d) the amino acid sequences shown in SEQ ID NO:14;
(e) the amino acid sequences shown in SEQ ID NO:15;
(f) the amino acid sequences shown in SEQ ID NO:16;
(g) the amino acid sequences shown in SEQ ID NO:17; and
(h) the amino acid sequences shown in SEQ ID NO:18.

42. A marker molecule of the formula I:
Segment A-L-Segment B
wherein,
Segment A is a labeled molecule;
L is a linker or a bond; and
Segment B is modified naturally occurring protein which contains a reduced number of post-translational modification sites.

43. The marker molecule of claim 40, wherein the post-translational modification sites are selected from the group consisting of:
(a) deamidation sites;
(b) glycosylation sites; and
(c) phosphorylation sites.

44. The marker molecule of claim 41, wherein in which contains none of one or more amino acid selected from the group consisting of:
(a) asparagine;
(b) glutamine;
(c) proline;
(d) serine;
(e) threonine;
(f) tyrosine; and
(g) aspartic acid.

45. A method of making a protein molecular marker, comprising:
eliminating one or more sequences encoding a first amino acid from the sequence of a nucleic acid molecule encoding a protein, wherein the protein comprises a particular number of sites for the direct or indirect attachment of a label, to generate a nucleic acid sequence encoding a recombinant protein having a reduced number of residues of the first amino acid;
expressing the recombinant protein;
isolating the recombinant protein; and
labeling the recombinant protein with a visibly colored chromophore or fluorophore.

46. The method of claim 45, wherein the protein comprises a repeated sequence.

47. The method of claim 45, wherein the first amino acid is cysteine.

48. The method of claim 45, wherein the first amino acid is lysine.

49. The method of claim 45, wherein labeling the protein is directly or indirectly attaching a label to one or more cysteine residues.

50. A method of making a protein molecular marker, comprising:
introducing one or more sequences encoding a first amino acid to the sequence of a nucleic acid molecule encoding a protein;
expressing the protein having incorporated residues of the first amino acid;
isolating the protein; and
labeling residues of the first amino acid of the protein with a visibly colored chromophore or fluorophore.

51. The method of claim 50, wherein the first amino acid is cysteine.

52. The method of claim 51, wherein labeling the protein is directly or indirectly attaching a label to one or more cysteine residues.

53. The method of claim 50, wherein the first amino acid is lysine.

54. The method of claim 53, wherein labeling the protein is directly or indirectly attaching a label to one or more lysine residues.

55. A composition comprising a collection of two or more marker molecules that differ in molecular weight and/or pI;
wherein the two or more marker molecules comprise one or more labeling molecules, wherein the labeling molecules comprise visibly colored chromophores or fluorophores; and
further wherein the two or more marker molecules contain no lysine residues or no cysteine residues, or the two or more marker molecules are recombinant proteins having a reduced number of lysine residues or a reduced of cysteine residues as compared with the wild type protein.

56. The composition of claim 55, wherein the two or more marker molecules are protein molecules that differ in pI.

57. The composition of claim 55, wherein the two or more marker molecules are protein molecules that differ in molecular weight.

58. The composition of claim 55, wherein the two or more marker molecules have molecular weights of from 3,000 daltons to 250,000 daltons.

59. The composition of claim 55, wherein the two or more marker molecules have a reduced number of cysteine residues as compared with the wild type protein.

60. The composition of claim 55, wherein the two or more marker molecules contain no cysteine residues.

61. The composition of claim 55, wherein the two or more marker molecules have a reduced number of lysine residues as compared with the wild type protein.

62. The composition of claim 55, wherein the two or more marker molecules contain no lysine residues.

63. The composition of claim 55, wherein at least one of the two or more marker molecules comprises a repeating sequence of amino acid residues.

64. The composition of claim 55, wherein the two or more marker molecules further comprise a tag.

65. The composition of claim 64, wherein the tag is a biotin, fluorescein, digoxigenin, or polyhistidine tag.


SEQUENCE LISTING (3 sequences)

SEQ ID NO: 1 - protein, 12 aa, Artificial
  Synthetic peptide
CKKRKKHHHHHH

SEQ ID NO: 2 - skipped
  Synthetic Peptide

SEQ ID NO: 3 - protein, 43 aa, Escherichia coli
MHTGSTTLPDFFAGMSDDFTPPIFAGYCRDDSHELRFRLYALL